(19) United States
(12) Patent Application Publication
(10) Pub. No.: US 2025/0288467 A1
(43) Pub. Date: Sep. 18, 2025

(54) ENGINEERED KNITTED GARMENT HAVING INTEGRATED COMPRESSION ZONES

(71) Applicant: Lululemon Athletica Canada Inc., Vancouver (CA)

(72) Inventors: Laura Flanagan, Vancouver (CA); Audrey Milligan Reilly, Vancouver (CA); Sophie Adrienne Doyle, Vancouver (CA); Alison Claire Maxfield, Halfmoon Bay (CA); Joshua David Hiney, San Francisco, CA (US); Sisi Jiang, Vancouver (CA); Yu Wen Angela Huang, Vancouver (CA); Eliot Jean Cohen Pirenne, Vancouver (CA); Alessia Isabella Atchison, Vancouver (CA); Clare Maree Robertson, Vancouver (CA); Lindsey Jean White, Vancouver (CA)

(21) Appl. No.: 19/220,999

(22) Filed: May 28, 2025

Related U.S. Application Data
(63) Continuation-in-part of application No. 18/970,181, filed on Dec. 5, 2024.
Application No. 18/970,181 is a continuation of application No. 17/006,177, filed on Aug. 28, 2020, now abandoned.
(60) Provisional application No. 62/894,292, filed on Aug. 30, 2019.

(51) Int. Cl. A61F 13/08; A41D 1/08

(52) U.S. Cl. CPC A61F 13/08; A41D 1/08; A41D 2500/10

(57) ABSTRACT

The present disclosure provides for engineered knitted garments multiple compression zones. The compressive moduli of the compression zones may be different to provide higher compression in areas where greater support is required and lower compression in areas requiring less support.

This application is a continuation-in-part of U.S. patent application Ser. No. 18/970,181, filed Dec. 5, 2024, entitled “ENGINEERED KNITTED GARMENT HAVING INTEGRATED COMPRESSION ZONES,” which is a continuation of U.S. patent application Ser. No. 17/006,177, filed Aug. 28, 2020, entitled “ENGINEERED KNITTED GARMENT HAVING INTEGRATED COMPRESSION ZONES,” now abandoned, which application claims the benefit of and priority to U.S. Provisional Application Ser. No. 62/894,292, filed Aug. 30, 2019, entitled “ENGINEERED KNITTED GARMENT HAVING INTEGRATED COMPRESSION ZONES,” the disclosures of which are incorporated herein by reference in their entireties.

### TECHNICAL FIELD

This application relates to the field of garments, and more specifically, to an engineered knitted garment having integrated compression zones for sporting activities, exercise, and the like.

### BACKGROUND

Athletes and consumers are demanding high performance activewear and sportswear, such as for example, compression sportswear. Studies have found that compression garments can provide benefits such as reducing muscle fatigue, providing muscle support, reducing muscle vibration, reducing the risk of strain, preventing injuries, etc. Currently, most compression garments are made by cutting and sewing fabrics with different structural properties to provide different levels of compression. However, such garments typically have a number of seams located throughout the garment that may cause chaffing. In addition, making such garments typically involves greater fabric waste and increased labor and manufacturing costs.

### SUMMARY OF THE APPLICATION

In one aspect, an engineered knitted garment such as a pair of tights is provided having at least a first compression zone located at the calves, a second compression zone located at the knees, and a third compression zone located at the waist. The compressive moduli of the compression zones may be different to provide higher compression in areas where greater support is required (e.g., at the calves) and lower compression in areas where greater mobility is required (e.g., at the knees).

In addition to the aspects and embodiments described above, further aspects and embodiments will become apparent by reference to the drawings and study of the following detailed description.

### BRIEF DESCRIPTION OF THE DRAWINGS

Throughout the drawings, reference numbers may be re-used to indicate correspondence between referenced elements. The drawings are provided to illustrate example embodiments described herein and are not intended to limit the scope of the application. Sizes and relative positions of elements in the drawings are not necessarily drawn to scale. For example, the shapes of various elements and angles may be not drawn to scale, and some of these elements may be arbitrarily enlarged and positioned to improve drawing legibility.

FIG. 1 is a front view illustrating an example engineered knitted garment having integrated compression zones.

FIG. 2 is a rear view thereof.

FIG. 3 is a front view thereof illustrating example compression zones.

FIG. 4 is a rear view thereof.

FIG. 5 is a left side view illustrating an example knitted panel for the engineered knitted garment of FIGS. 1-4.

FIG. 6 is a front view illustrating another example engineered knitted garment having integrated mesh texture compression zones.

FIG. 7 is a rear view thereof.

FIG. 8 includes left, right, front, and rear views illustrating example knitted panels for the engineered knitted garment of FIGS. 6-7.

FIG. 9 is a block diagram illustrating an example warp knitting machine.

FIG. 10 is a front view illustrating another example engineered knitted garment having integrated compression zones.

FIG. 11 is a rear view thereof.

FIG. 12 is a front view illustrating another example engineered knitted garment having integrated compression zones.

FIG. 13 is a rear view thereof.

FIG. 14 is a front view illustrating another example engineered knitted garment having integrated compression zones.

FIG. 15 is a front view illustrating another example engineered knitted garment having integrated compression zones.

FIG. 16 is a front view illustrating another example engineered knitted garment having integrated compression zones.

FIG. 17 is a rear view thereof.

FIGS. 18-22 are front, side, and rear views illustrating additional example engineered knitted garments having integrated compression zones.

FIGS. 23A and 23B illustrate the front and rear views of a torso garment that is sized and shaped to be worn over the upper torso area.

FIGS. 24A and 24B illustrate the front and rear views of a torso garment that is sized and

shaped to be worn over the upper and mid-torso areas.

FIGS. 25A and 25B illustrate the front and rear views of a torso garment that is sized and shaped to be worn over the upper, mid, and lower torso areas.

FIGS. 26A and 26B illustrate the front and rear view of a torso garment that is sized and shaped to be worn over the upper, mid, and lower torso and also is sized and shaped to be worn over the upper to mid-thigh areas.

FIG. 27A illustrates the inner surface of the front of an upper torso garment. FIG. 27B illustrates various positions for a slit.

FIG. 28A illustrates the inner surface of the front of an upper torso garment. FIG. 28B illustrates various positions for a slit.

FIG. 29 illustrates the inner surface of the front of an upper torso garment.

### DETAILED DESCRIPTION

In the following description, details are set forth to provide an understanding of the application. In some instances, certain structures, techniques, and methods have not been described or shown in detail in order not to obscure the application.

The warp knitting process involves making a series of vertical columns of connected stitches (ground stitches) by forming a series of interconnected loops between a series of yarns (ground yarns). In a warp knit, the vertical columns of connected stitches are referred to as wales, and the wales run parallel to the length of the fabric or component. While forming the ground stitches, one or more additional yarns may be inserted into or between the ground stitches. The inserted yarns are sometimes referred to as the weft yarns, as in the warp knit structure, the inlaid yarns run in a direction generally perpendicular to the direction of the courses of ground stitches. The inlaid yarns can be used to form patterns in the warp knitted fabric or component. In a warp knitting machine, a stationary needle bed and moving guide bars are used to form the ground stitches. Conventional guide bars typically are configured to guide the yarns feeding several needles at a time and are limited in the extent of their movements. Warp knitting machines may also include one or more jacquard guide bars. Jacquard guide bars allow more complex patterns to be made as they allow the position of a single yarn (either a ground yarn or an inlaid yarn) to be varied on a needle-by-needle basis, allowing what is referred to as jacquard movement during the knitting process, and allowing the creation of jacquard stitch constructions. Typically, illustrations of knit structures which employ jacquard guide bars include indications of where jacquard movement occurs during the knitting process.

In jacquard warp knitting machines having a single jacquard guide bar, the single jacquard guide bar is used to control the ground yarns, allowing the ground pattern to be more complex (i.e., a jacquard ground pattern or jacquard ground stitch construction with or without one or more additional yarns being inserted into or between the ground stitches). In jacquard warp knitting machines having two jacquard guide bars, the first jacquard guide bar may be used to control the ground yarns and the ground pattern in the same manner as when a single jacquard guide bar knitting machine is used to create a jacquard ground pattern, while the second jacquard guide bar may be used to independently create a second pattern of an inlaid yarn and add the second pattern to the ground pattern. In other words, one of the guide bars may be used to provide a desired combination of stitches and knit constructions while the other guide bar may be used to add extra yarn (e.g., jacquards in elastane) to change the density, the fabric weight, or both the density and the fabric weight in a particular zone of a warp knitted fabric or component. Using the two jacquard guide bars in this manner results in a warp knitted fabric or component having a desired combination of stitches and knit constructions which include both a jacquard ground pattern (i.e., a jacquard ground knit construction) and an added jacquard pattern (i.e., an added jacquard stitch construction). Optionally, the added jacquard pattern may include one or more added yarns which are different than the ground yarns used in the ground (i.e., first) jacquard pattern. Whether or not the yarns used in the added (i.e., second) jacquard pattern are the same as or different from the ground yarns, the use of the second jacquard guide bar allows an additional amount (e.g., an additional volume or weight) of yarn to be inlaid in the added (i.e., second) jacquard stitch construction, and the added (i.e., second) jacquard stitch construction is formed and added concurrently with the formation of the ground (i.e., first) stitch construction. The use of two jacquard guide bars in this manner results in a warp knitted fabric or component having a knit structure including both a jacquard ground pattern (i.e., a jacquard ground stitch construction) made using the first jacquard guide bar and an added jacquard pattern (i.e., an added jacquard stitch construction) made using the second jacquard guide bar. The addition of the added (i.e., second) jacquard pattern to the ground (i.e., first) jacquard pattern increases the complexity of the knit structure of the warp knitted fabric or component and can increase its compression level or modulus, density and fabric weight depending upon the type and amount of yarn or yarns used in the added jacquard pattern. The use of two jacquard guide bars in this manner also allows the creation of multiple compression zones which are connected by seamless transitions. In one aspect, the addition of the second jacquard pattern to the ground jacquard pattern can increase the fabric weight in a compression zone of a fabric or component by an amount ranging from about 20 grams per square meter (GSM) to about 220 GSM or from about 100 GSM to about 220 GSM as compared to the fabric weight of a compression zone with the ground jacquard pattern alone.

While using an elastomeric yarn such as an elastane yarn in a ground jacquard pattern can provide stretch and recovery to the ground jacquard pattern portion of a knit construction of a warp knitted fabric or component, using an elastane yarn in the added jacquard pattern (i.e., adding an elastane jacquard pattern or stitch construction) to the ground jacquard pattern by using a different (e.g., second) jacquard guide bar than is used to make the ground jacquard pattern provides a number of additional advantages. Adding in an elastane jacquard stitch construction (i.e., an elastane jacquard construction) using a second jacquard guide bar can increase the total amount of elastane yarn that can be incorporated into a compression zone, thereby increasing the compression level, compression modulus, density, fabric weight, or any combination thereof, of the compression zone as compared to the ground jacquard pattern alone. A level of compression provided by a garment or a portion of a garment (e.g. a compression zone) can be quantified in millimeters of mercury (mmHg) and can range from mild compression (8-15 mmHg) to very strong compression (40-50 mmHg). In the garments described herein, the level of compression can range from about 1 mmHg to about 50 mmHg, or from about 2 mmHg to about 40 mmHg, or from about 5 mmHg to about 30 mmHg. The test method ISO 20932 is commonly used to determine the modulus and extension of fabric and can be used to evaluate the compression level (e.g., the compression modulus) of a compression zone. Adding in an elastane jacquard stitch construction using a second jacquard guide bar can allow a different elastane yarn to be used in the elastane jacquard than the elastane yarn used in the ground pattern of a compression zone. Using an elastane jacquard construction added to a ground jacquard pattern by a second jacquard guide bar can allow multiple sizes or types of elastane yarns to be used in the elastane jacquard construction of a compression zone. By adding an elastane jacquard construction to a ground jacquard pattern using a second jacquard guide bar, it is possible to vary the elastane jacquard constructions across compression zones while keeping the ground jacquard pattern the same across the compression zones. Alternatively, both the ground jacquard patterns and the elastane jacquard constructions can be varied across compression zones. Using a second jacquard guide bar to add elastane jacquard constructions to ground jacquard patterns, the shape of the added compression zone is not limited to vertical or horizontal lines as the elastane need not be added in the wales of the ground stitches (into or between the ground stitches) but rather can be added as a jacquard pattern (i.e., elastane jacquard construction). Further, adding elastane jacquard constructions to ground jacquard patterns using a second jacquard guide bar creates seamless transitions between compression zones and does not require altering the ground jacquard pattern between compression zones, although the ground jacquard pattern can also be varied between compression zones while still creating seamless transitions.

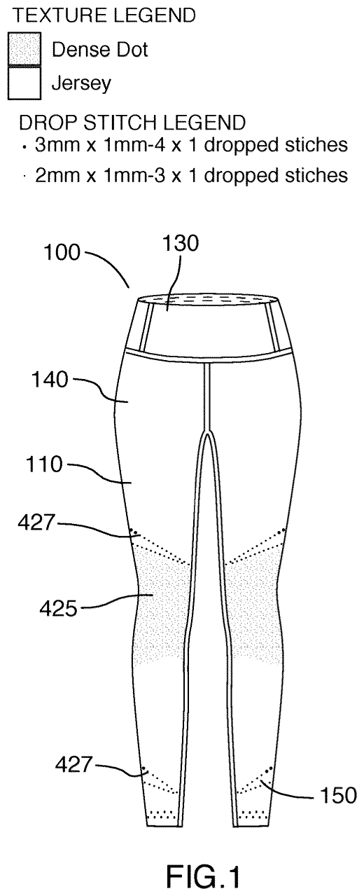

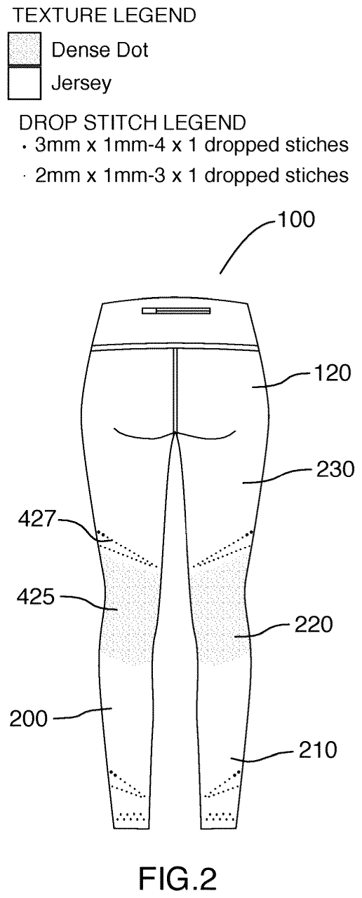

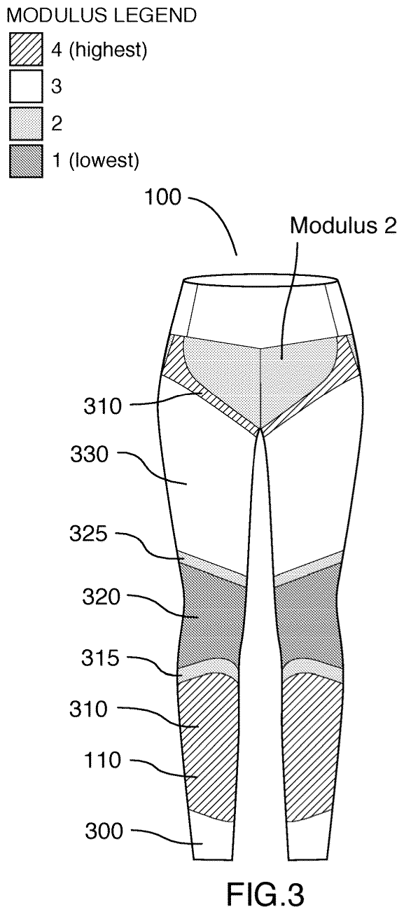

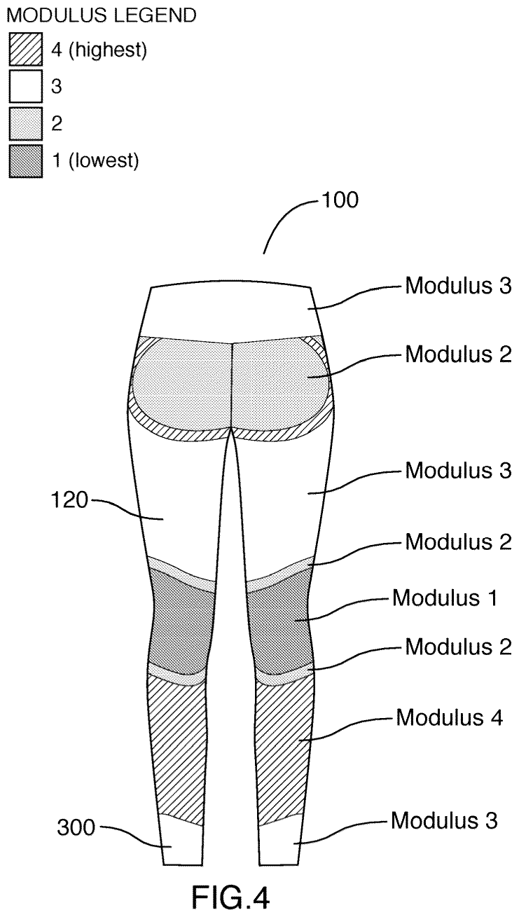

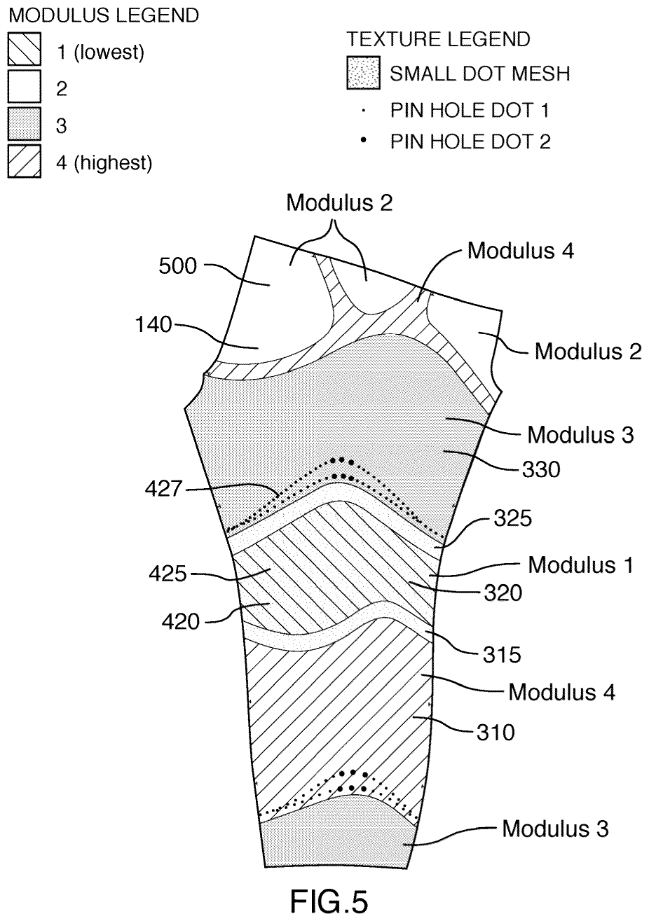

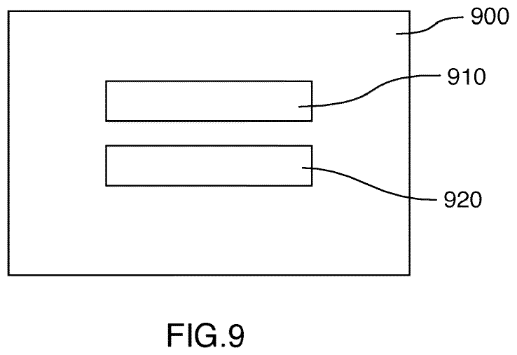

FIG. 1 is a front view illustrating an example engineered knitted garment 100 having integrated compression zones 300. FIG. 2 is a rear view thereof. FIG. 3 is a front view thereof illustrating example compressive zones 300. FIG. 4 is a rear view thereof. FIG. 5 is a left side view of an example knitted panel 500 for the engineered knitted garment 100 of FIGS. 1-4. And, FIG. 9 is a block diagram illustrating an example warp knitting machine 900.

According to one embodiment of the application, there is provided an engineered knitted garment 100 having integrated compression zones 300. The garment 100 may be a compression tight or pair of tights. Of course, the garment 100 may be any other garment such as a bra, top, shirt, jacket, skirt, skort, pants, shorts, bathing suits, etc. The garment 100 has a front side 110 and a rear side 120, a waistband 130, and first and second or left and right legs 140, 150. Each leg 140, 150 of the garment 100 may be formed from a knitted panel 500 (such as the left knitted panel 500 shown in FIG. 5).

The knitted panel 500 is shaped and sized to cover a portion of a wearer's body (e.g., the wearer's leg, a portion of the wearer's torso, etc.) and may be knitted using a raschel knitting machine 900 having two jacquard guide bars 910, 920. An example of such a machine is the Rascheltronic™ 4/2 jacquard raschel warp knitting machine available form Karl Mayer Holding GmbH & Co. KG. The two jacquard guide bars 910, 920 are used for seamlessly knitting different compression zones 300. One of the guide bars (e.g., 910) may be used to provide a desired combination of stiches and knit constructions 200 while the other guide bar (e.g., 920) may be used to adds extra yarn (e.g., jacquards in elastane) to change the density and/or weight in a particular zone such that the weight difference between the lowest compression zone and the highest compression zone may be around 200 grams per square meter (GSM). Typically, this cannot be obtained with a single jacquard bar knitting machine. Different levels (e.g., 3 or 4) of elastane content (or similar synthetic fibers or yarn such as Spandex™, Lycra™, polyamide, polyether-polyurea copolymer, etc.) may be engineered into the fabric of the knitted panel 500 to create different compression zones (e.g., 3 or 4) 300. Mesh textures 425 for ventilation zones 420 may also be included. The mesh textures 425 may be used to provide lower compression zones 300 in selected areas. Further compression zones 300 may be created by combining elastane jacquard with jacquard patterns which provides a double effect with respect to compression.

According to one embodiment, the knitted panel 500 may include a first compression zone 310 having a first knit stitch construction (or pattern) (e.g., Jersey knit, etc.) 210. One of the jacquard guide bars 910 of the knitting machine 900 may be used to define the first knit stitch construction 210 and the other of the jacquard guide bars 920 may be used to add an elastane jacquard construction (or pattern) to the first knit stitch construction 210 to increase fabric weight in the first compression zone 310. The first compression zone 310 may have a first amount of compression (or compressive modulus).

The knitted panel 500 may include a second compression zone 320 joined to the first compression zone 310 by a seamless transition 315 and may have a second knit stitch construction (or pattern) (e.g., Dense Dot knit, etc.) 220. The second compression zone 320 may have a second amount of compression (or compressive modulus). According to one embodiment, the first amount of compression of the first compression zone 310 may be greater than the second amount of compression of the second compression zone 320. That is, the first compression zone 310 may provide a tighter or more compressive fit that the second compression zone 320. Advantageously, the seamless joining of the first and second compression zones 310, 320 reduces chaffing.

According to one embodiment, a difference in fabric weight between the first compression zone 310 and the second compression zone 320 may be between 100 and 220 grams per square meter (GSM). According to one embodiment, the first and second knit stitch constructions 210, 220 of the garment 100 may be the same. According to one embodiment, at least one of the first and second knit stitch constructions 210, 220 may be a jacquard pattern.

For example, the second knit stitch construction 220 may include an elastane jacquard construction (or pattern) that is added to the second knit stitch construction 220 using the other of the jacquard guide bars. According to one embodiment, an amount (or extent) of the elastane jacquard in the first knit stitch construction 210 may be greater than an amount (or extent) of the elastane jacquard in the second knit stitch construction 220.

According to one embodiment, the engineered knitted garment 100 may further include a third compression zone 330 joined to at least one of the first compression zone 310 and the second compression zone 320 by a respective seamless transition 325 and having a third knit stitch construction (or pattern) (e.g. Jersey knit, etc.) 230. One of the jacquard guide bars of the knitting machine may be used to define the third knit stitch construction 230. The third compression zone 330 may have a third amount of compression (or compressive modulus). According to one embodiment, the third amount of compression of the third compression zone 330 may be greater than the second amount of compression of the second compression zone 320 but less than the first amount of compression of the first compression zone 310. The third knit stitch construction 230 may be a jacquard pattern. The third compression zone 330 may include an elastane jacquard construction (or pattern) added to the third knit stitch construction 230 using the other of the jacquard guide bars. According to one embodiment, an amount (or extent) of the elastane jacquard in the third compression zone 330 may be less than an amount of elastane jacquard in the first compression zone 310.

According to one embodiment, at least a portion of the second compression zone 320 may include a mesh texture or pattern 425. According to another embodiment, the garment 100 may further include at least one ventilation zone 420 seamlessly knitted into the knitted panel 500. The ventilation zone 420 may be formed from or include a mesh 425 or a pattern of drop stitch open holes 427. Advantageously, sheer and open hole mesh textures 425, 427 may be knit into the knitted panel 500 to improve breathability, to achieve a lower compressive modulus, to reduce restriction, and/or for visual effect.

As mentioned above, according to one embodiment as shown in FIGS. 1-4, the engineered knitted garment 100 may be a compression tight (or pair of compression tights or tights). In this embodiment, the first compression zone 310 may be located in one or more of a calf area, an under buttock area, and an upper waistband area with anchoring portions to respective hip areas of the compression tight 100. The second compression zone 320 may be located in a knee area of the compression tight 100. The garment 100 may further include a third compression zone 330 joined to at least one of the first compression zone 310 and the second compression zone 320 by a respective seamless transition 325 and having a third knit stitch construction 230 wherein one of the jacquard guide bars is used to define the third knit stitch construction 230, the third compression zone 330 having a third amount of compression (or compressive modulus), the third amount of compression being greater than the second amount of compression of the second compression zone 320 but less than the first amount of compression of the first compression zone 310. The third compression zone 330 may be located in one or more of a pelvic area and an area at least partially surrounding a knee area of the compression tight 100. Advantageously, the compressive moduli of the compression zones 300 may be different to provide higher compression in areas where greater support is required (e.g., at the calves) and lower compression in areas where greater mobility is required (e.g., at the knees). The high modulus in the under buttock and upper waistband areas anchors the compression tight 100 to the waistband and reduces crotch sagging and bum jiggle. The low modulus in the knee area of the compression tight 100 reduces restriction and bulk. And, the gradate levels of compression in the transition 325 provide a distraction free fit.

In some embodiments, the garment 100 may have more than three compression zones 300.

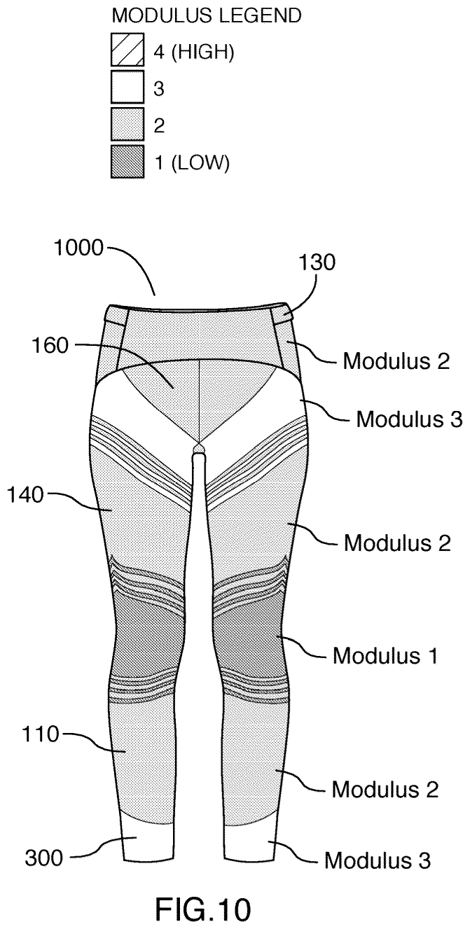

For example, FIGS. 3 and 4 illustrate a garment 100 (e.g., a pair of tights) having 4 compression zones. As described further below, FIGS. 10 to 13 also illustrate garments 1000, 1200 having 4 compression zones.

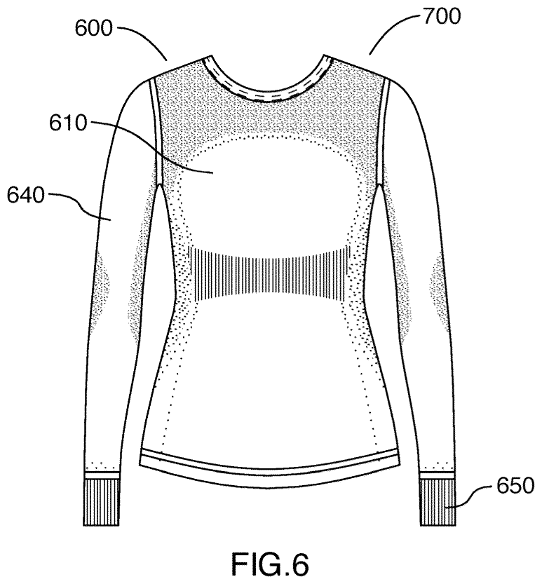

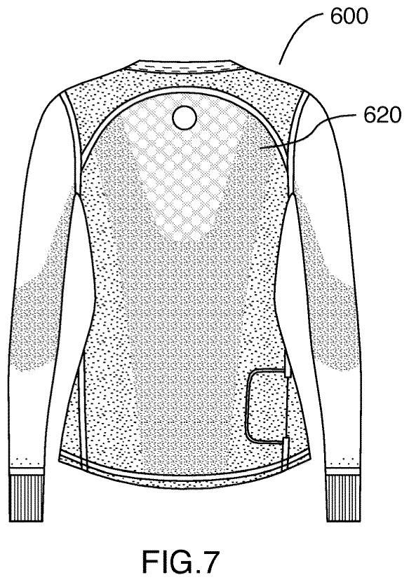

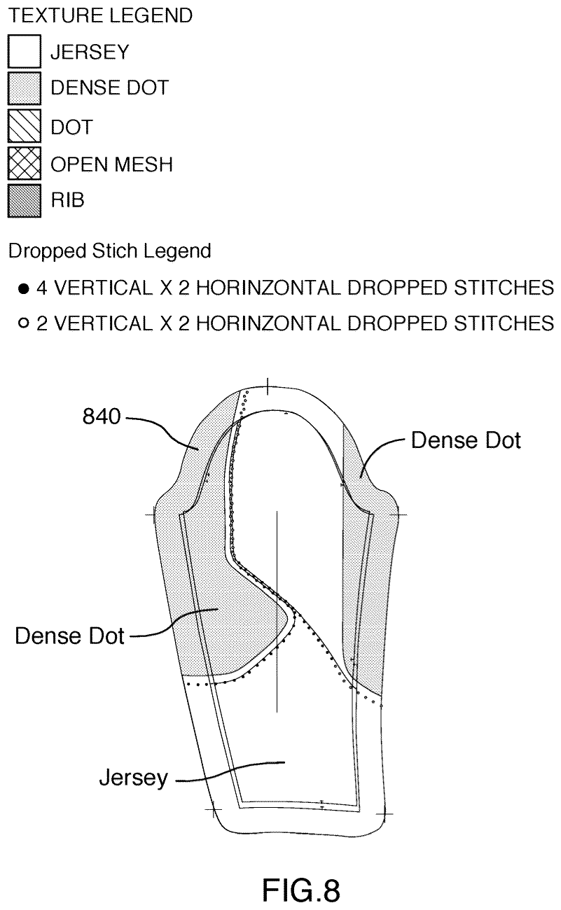

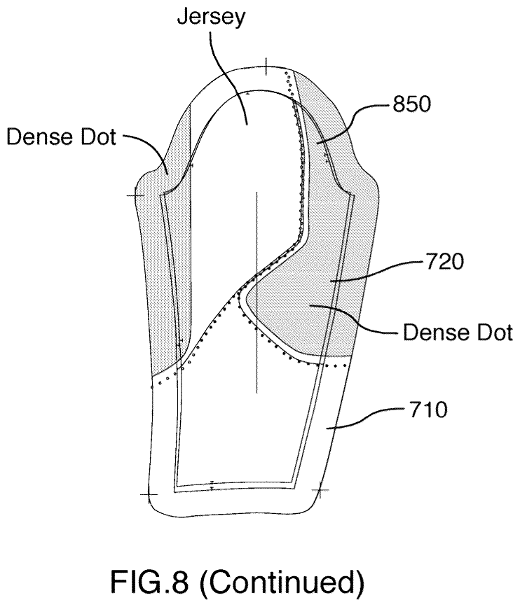

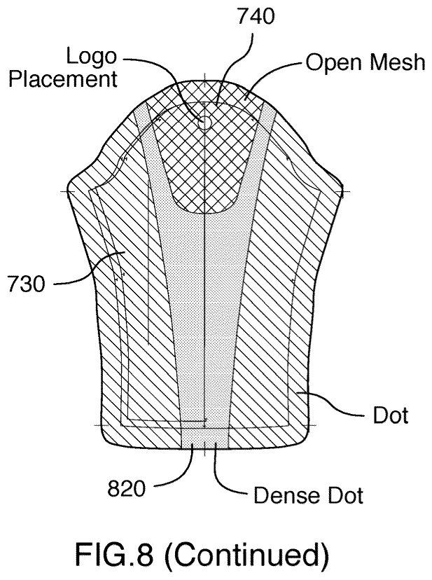

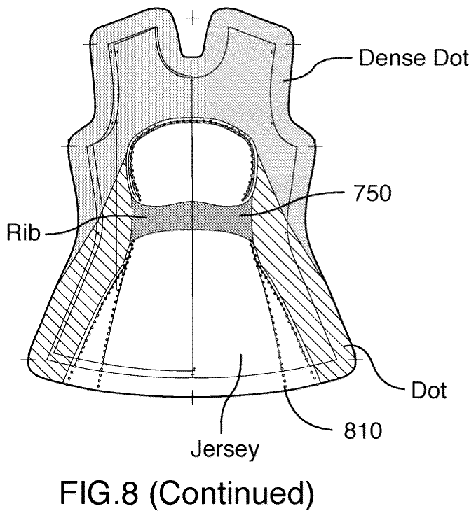

FIG. 6 is a front view illustrating another example engineered knitted garment 600 having integrated mesh texture compression zones 700. FIG. 7 is a rear view thereof. And, FIG. 8 includes left, right, front, and rear views illustrating example knitted panels 800 for the engineered knitted garment 600 of FIGS. 6-7.

As mentioned above, the engineered knitted garment of the present application may be any garment such as a bra, top, shirt, jacket, skirt, skort, pants, shorts, bathing suits, etc., in addition to the tights 100 shown in FIGS. 1-4. For example, FIG. 6 shows an engineered knitted garment 600 which is a top or shirt. The garment 600 has a front side 610, a rear side 620, and first and second or left and right arms 640, 650. Each arm 640, 650 and the front and rear sides 610, 620 of the garment 600 may be formed from a respective knitted panel 800 (such as the knitted panels 810, 820, 840, 850 shown in FIG. 8).

The garment 600 has multiple compression zones 700 that are formed from different mesh textures. The different mesh textures provide different levels of breathability (or ventilation) and different levels (or moduli) of compression. For example, the wrist area of each arm 640, 650 and the lower area on the front side 610 of the garment 600 may have a jersey texture providing a first compression zone 710. The elbow area of each arm 640, 650, the under neck area on the front side 610, and the middle back area on the rear side 620 of the garment 600 may have a dense dot texture providing a second compression zone 720. The underarm areas on the sides of the rear side 620 of the garment 600 may have a dot texture providing a third compression zone 730. The upper back area on the rear side 620 of the garment 600 may have an open mesh texture providing a fourth compression zone 740. And, the middle area on the front side 610 of the garment 600 may have a rib texture providing a fifth compression zone 750. For example, the first compression zone 710 (at the wrists) may have a higher compressive modulus than the second compression zone 720 (at the elbows) to improve fit and user comfort.

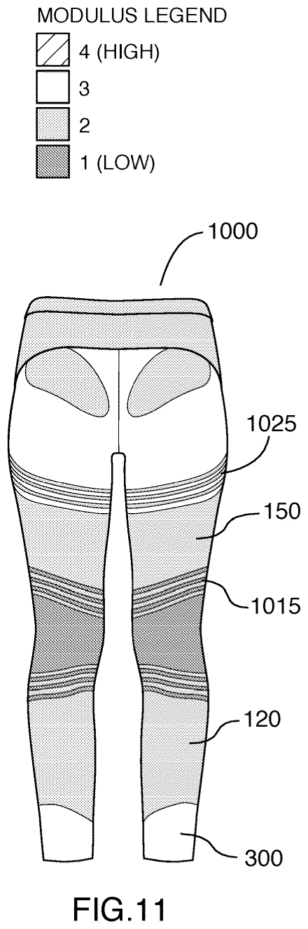

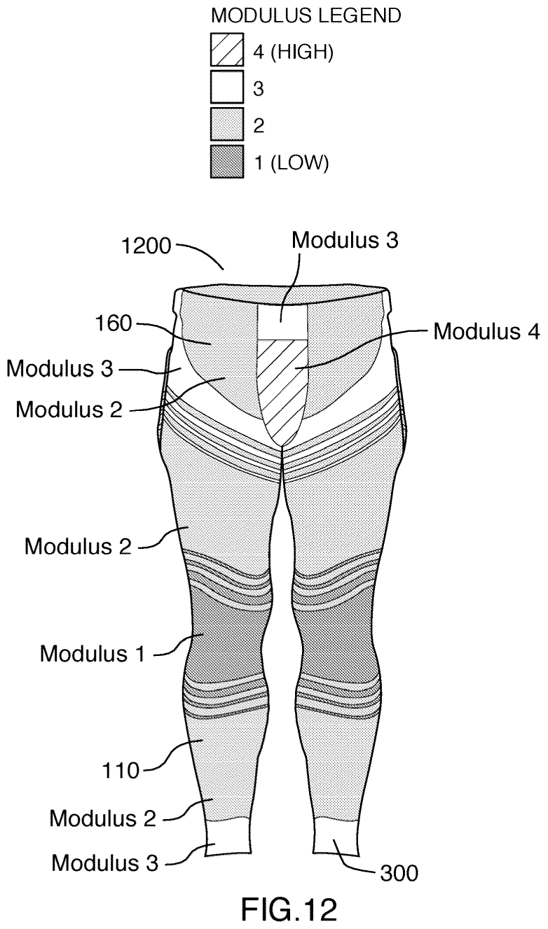

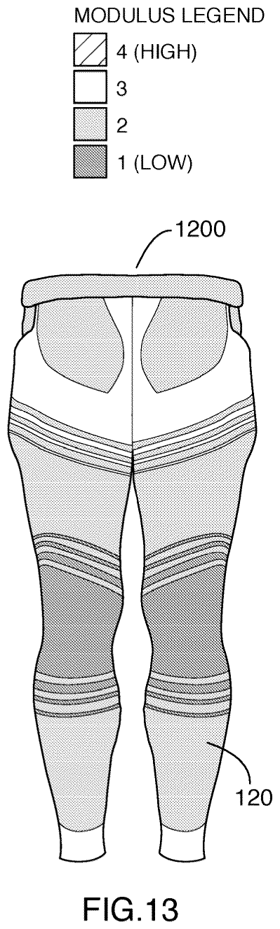

FIG. 10 is a front view illustrating another example engineered knitted garment 1000 having integrated compression zones 300. FIG. 11 is a rear view thereof. FIG. 12 is a front view illustrating another example engineered knitted garment 1200 having integrated compression zones 300. And, FIG. 13 is a rear view thereof. Similar to the engineered knitted garment 100 of FIGS. 3-4, the engineered knitted garments 1100, 1200 of FIGS. 10-11 and FIGS. 12-13 may be compression tights or pairs of tights for women and men, respectively. The garments 1100, 1200 each have a front side 110, a rear side 120, a waistband 130, and first and second or left and right legs 140, 150. The garments 1100, 1200 include a number (e.g., 4) of compression zones 300 each having a respective amount of compression (or compressive modulus) (e.g., Modulus 1, Modulus 2, etc.). The layout or arrangement of the compression zones 300 in the pelvic area 160 of the garments 1100, 1200 is tuned to match the sex of the wearer to provide support for sensitive areas. For example, and referring to FIG. 10 which shows the women's garment 1000, less support (i.e., a lower compressive modulus Modulus 2) may be provided in the genital area and greater support (i.e., a higher compressive modulus Modulus 3) may be provided around the genital area. As another example, and referring to FIG. 12 which shows the men's garment 1200, greater support (i.e., a higher compressive modulus Modulus 4) may be provided in the genital area and less support (i.e., a lower compressive modulus Modulus 2) may be provided around the genital area. The seamless transitions 1015, 1025 between compression zones 300 may be formed by alternating bands (e.g., 3 bands) of fabric from each of the adjoining compression zones 300. The garment 1100, 1200 may be of light weight being made from, for example, lighter 60 dtex elastane rather than, for example, the heavier 78 dtex elastane that may be used in the garment 100 of FIGS. 3-4. This construction is advantageous for use in running activities.

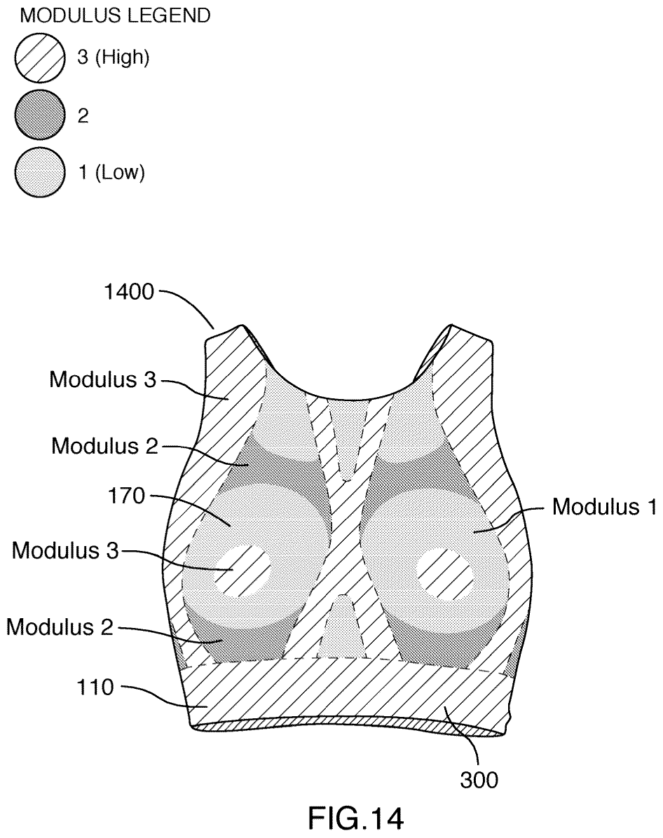

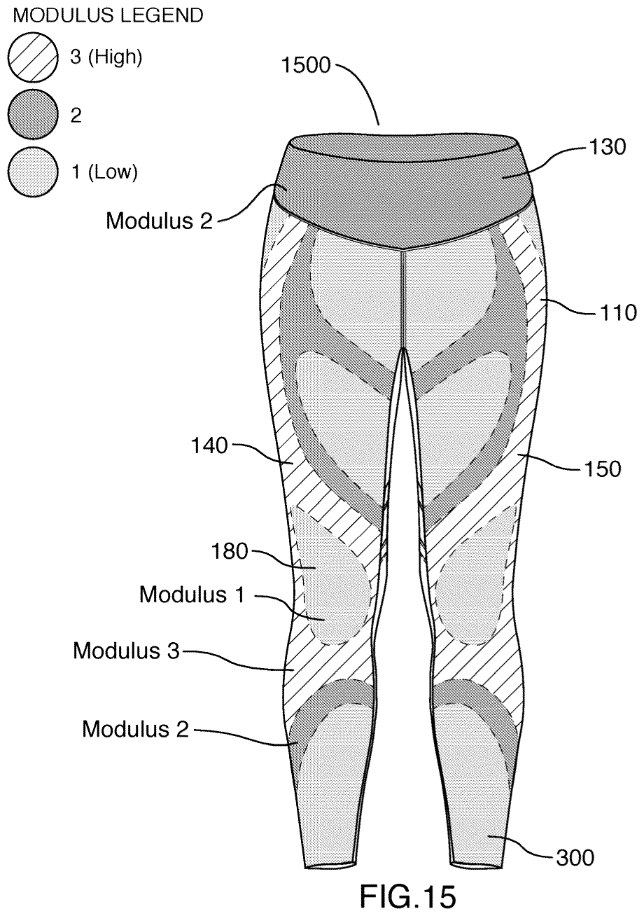

FIG. 14 is a front view illustrating another example engineered knitted garment 1400 having integrated compression zones 300. And, FIG. 15 is a front view illustrating another example engineered knitted garment 1500 having integrated compression zones 300. The engineered knitted garment 1400 of FIG. 14 may be a sports bra or top and the engineered knitted garment 1500 of FIG. 15 may be compression tights or a pair of tights. The garments 1400, 1500 include a number (e.g., 3) of compression zones 300 each having a respective amount of compression (or compressive modulus) (e.g., Modulus 1, Modulus 2, etc.). The layout or arrangement of the compression zones 300 in the breast area 170 of the garment 1400 of FIG. 14 is tuned to provide support for sensitive areas. For example, greater support (i.e., a higher compressive modulus Modulus 3) may be provided in the areola/nipple area, and on either side of the breast tissue, and less support (i.e., a lower compressive modulus Modulus 1) may be provided around the areola/nipple area.

The layout of the compression zones 300 in the joint areas 180 (e.g., in the knee area) of the garment 1500 of FIG. 15 is tuned to provide greater support (i.e., a higher compressive modulus Modulus 3) around the joint and less support (i.e., a lower compressive modulus Modulus 1) at the joint. This provides the wearer with an increased feeling of support and awareness in the joint areas 180. Such garments may be used in training activities where additional support around the joints is desired. The garments 1400, 1500 may be of light weight and may be made using cooling yarn which is advantageous for use in training activities.

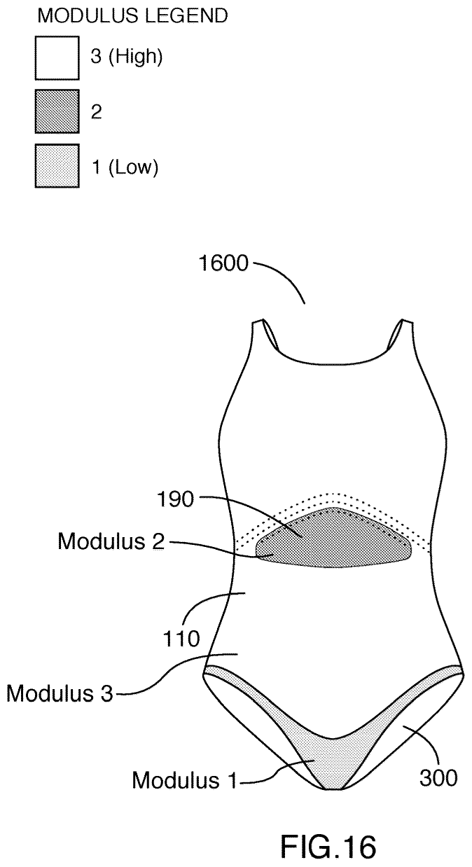

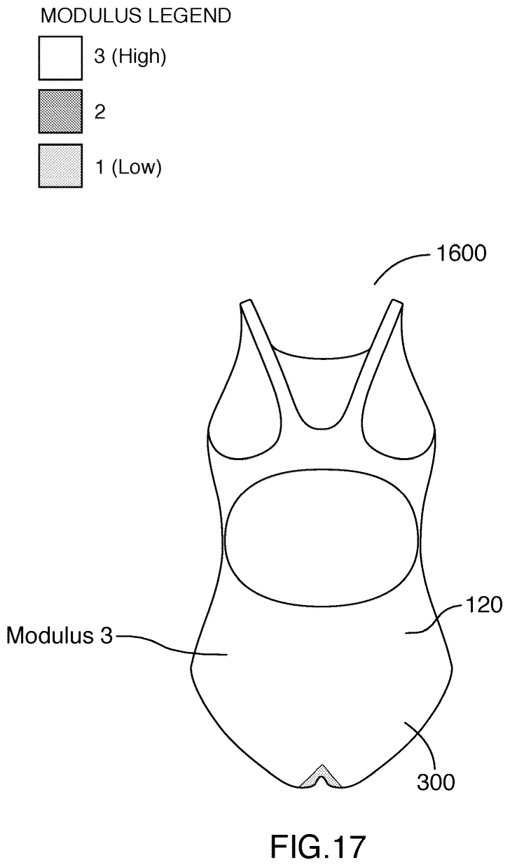

FIG. 16 is a front view illustrating another example engineered knitted garment 1600 having integrated compression zones 300. And, FIG. 17 is a rear view thereof. The engineered knitted garment 1600 of FIGS. 16-17 be a bathing suit or leotard. The garment 1600 includes a number (e.g., 3 or 4) of compression zones 300 each having a respective amount of compression (or compressive modulus) (e.g., Modulus 1, Modulus 2, etc.). The layout or arrangement of the compression zones 300 in the abdominal area 190 of the garment 1600 of FIG. 16 is tuned to provide support where required. For example, to improve flexibility, less support (i.e., a lower compressive modulus Modulus 2) may be provided in the central area of the abdomen and greater support (i.e., a higher compressive modulus Modulus 3) may be provided around the central area.

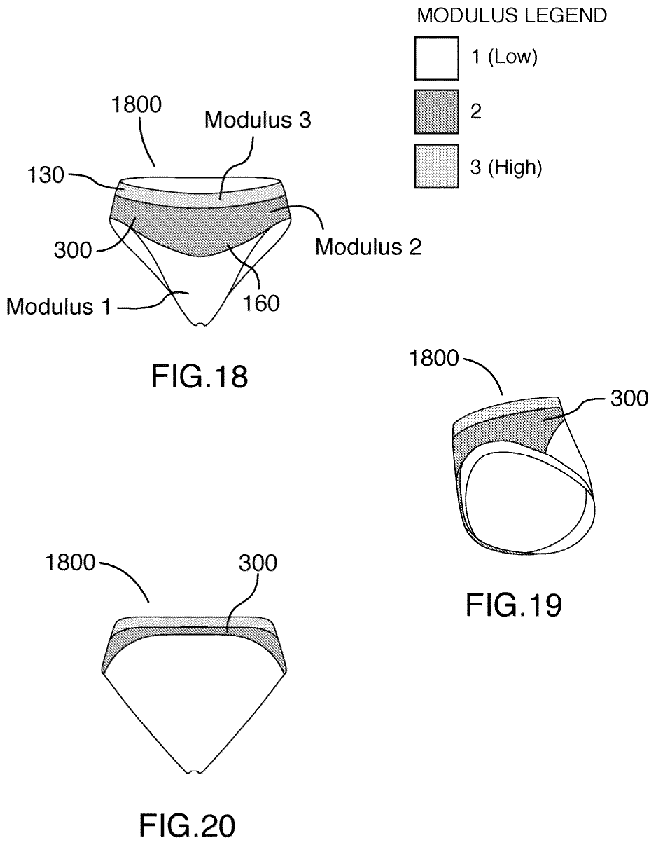

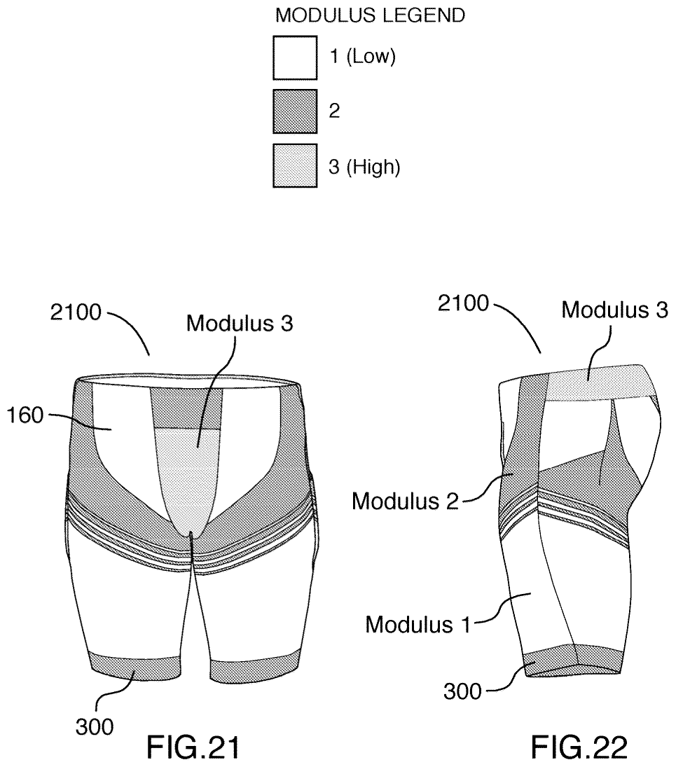

FIGS. 18-22 are front, side, and rear views illustrating additional example engineered knitted garments 1800, 2100 having integrated compression zones 300. The engineered knitted garments 1800, 2100 of FIGS. 18-20 and FIGS. 21-22 may be underwear for women and men, respectively. The garments 1800, 2100 include a number (e.g., 3 or 4) of compression zones 300 each having a respective amount of compression (or compressive modulus) (e.g., Modulus 1, Modulus 2, etc.). Similar to the garments 1100, 1200 of FIGS. 10-13, the layout or arrangement of the compression zones 300 in the pelvic area 160 of the garments 1800, 2100 is tuned to match the sex of the wearer to provide support where required for sensitive areas. In addition, and referring to FIGS. 18-20 which show the women's garment 1800, greater support (i.e., a higher compressive modulus Modulus 3) may be provided in the waistband area or waistband 130 to replicate or approximate the hugged feeling provided by a pair of compression tights and less support (i.e., a lower compressive modulus Modulus 2) may be provide in the pelvic area 160 below the waistband 130.

In some embodiments, the compression zones 300 may be made from a blend of natural and synthetic fabrics.

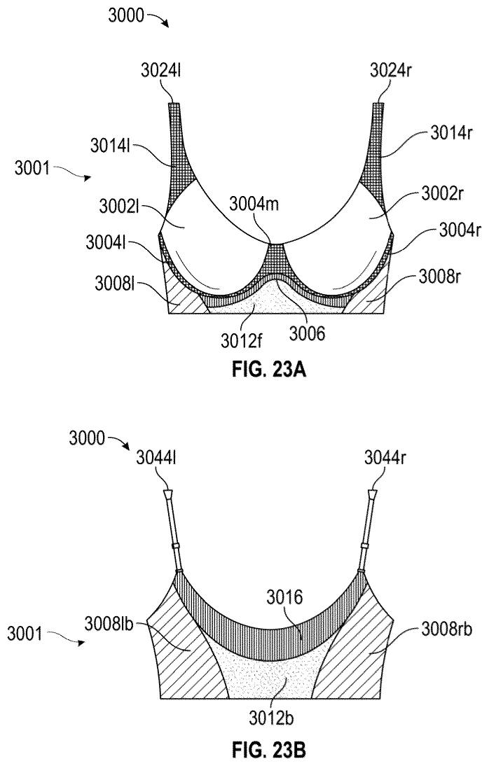

FIGS. 23A-29 will now be described. In general, FIGS. 23A-29 describe garments (e.g., garments 3000, 4000, 5000, 6000, 8000a, 8000b, 9000) that are configured to be worn over the upper torso area (also referred to as an “upper torso” garment), the upper and mid-torso areas (also referred to as a “half torso” garment), the upper, mid-, and lower torso areas (also referred to as a “full torso” garment), or the upper, mid-, lower torso and thigh areas (e.g., upper to mid-thigh areas) (also referred to as a “full torso garment with shorts” or “bodysuit with shorts”). The garments described and illustrated in FIGS. 23A-29 can comprise a single engineered warp knitted component (3001, 4001, 5001, 6001, 8001a, 8001b, 9001) (e.g., combined with one or more additional components such as separate strap component or at least one liner or both separate strap components and at least one liner), or can consist of only the single engineered warp knitted component (3001, 4001, 5001, 6001, 8001a, 8001b, 9001).

In an aspect, the garments are shaper garments. In an aspect, a garment can be a “shaper” garment in the present disclosure if the garment includes one or more compression areas (e.g., one or more areas having an amount of compression or compression modulus). The garments illustrated in FIGS. 23A-29 are shaper garments.

In an aspect, the garments can include a bra, a bodysuit (e.g., a bodysuit brief, bodysuit thong-cut, bodysuit boy shorts), a shirt, a tank top, a camisole, or a slip, where one or more areas of the garment include a compression area. Each of the illustrated garments comprises a single engineered warp knitted component (3001, 4001, 5001, 6001, 8001a, 8001b, 9001). Additional components such as elasticated components, strap adjustment components, snaps, hooks, and the like may be affixed to the single engineered warp knitted component. (3001, 4001, 5001, 6001, 8001a, 8001b, 9001) The components may be affixed by adhesive bonding, thermal bonding, stitching, and any other means known in the art.

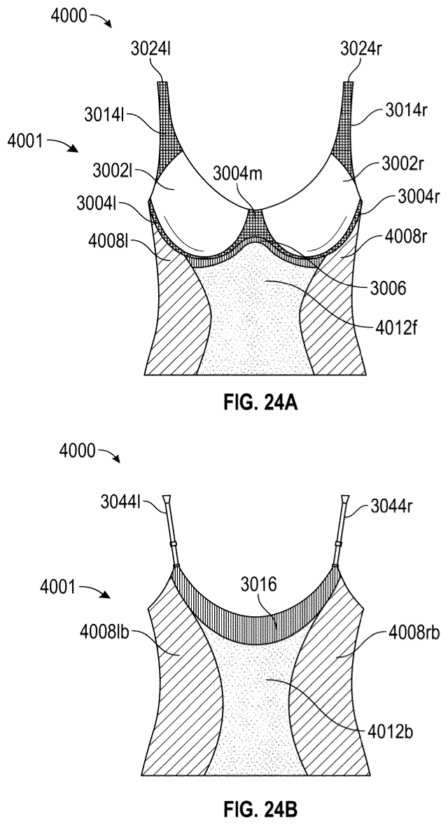

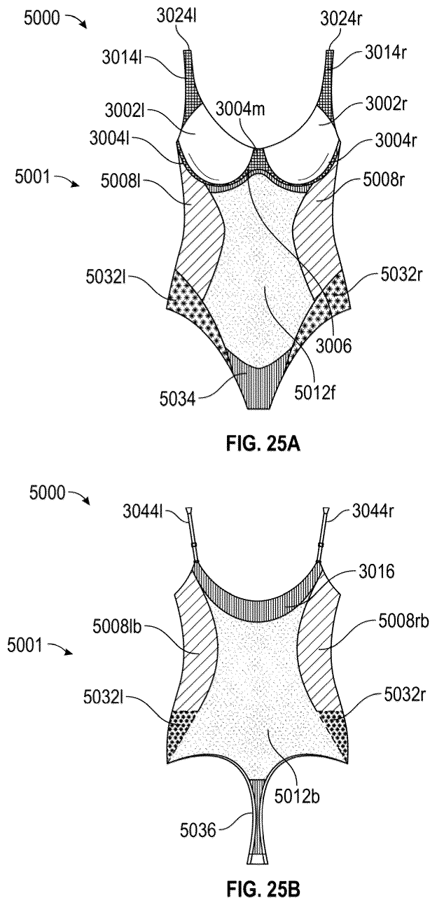

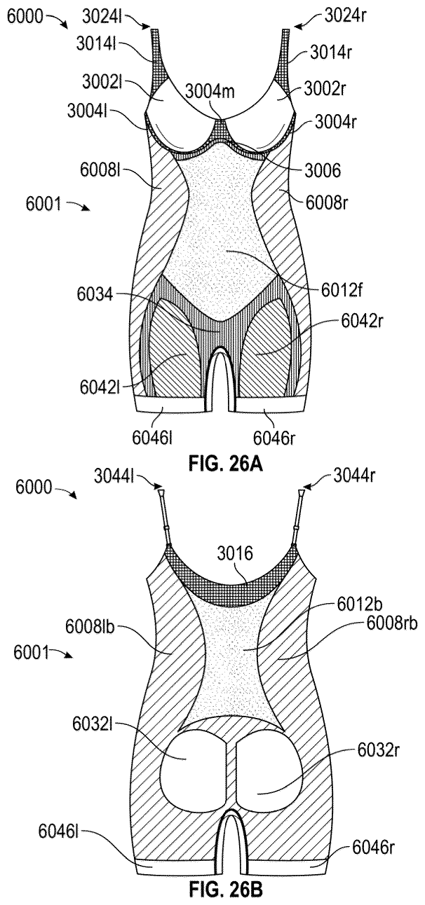

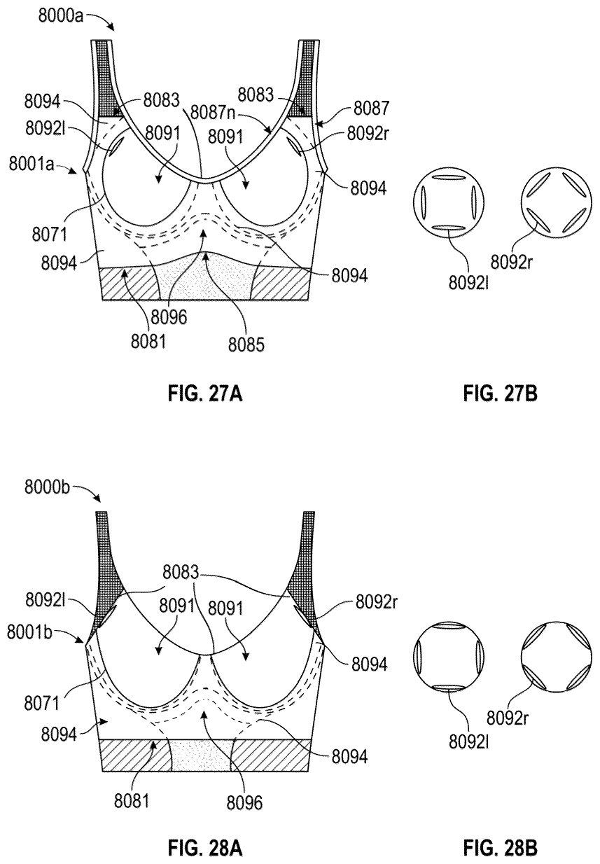

FIGS. 23A and 23B illustrate an upper torso garment that is a bra. FIGS. 24A and 24B illustrate a half torso garment that can be a shirt, a tank top, or a camisole. FIGS. 25A and 25B illustrate a full torso garment that can be a bodysuit with a thong-cut bottom portion, but the full torso garment can be a bodysuit that includes a bottom portion which is not thong-cut and is sized and shaped to cover at least a portion of the upper thigh and buttocks areas, such as a bikini-cut or shorts-cut bottom portion. FIGS. 26A and 26B illustrate a full torso garment that is sized and shaped to cover the upper to mid-thigh areas, which can be a bodysuit with a shorts-cut bottom portion (where the shorts can be referred to as “boy shorts”). In some examples, the full torso garment can have an inseam length of the shorts portion of 1 inch, 2 inches, 3 inches, 4 inches, 5 inches, 6 inches, etc. FIGS. 27A-29 illustrates upper torso garments, where the upper torso garment includes a liner that can be sized and shaped to provide extra support and coverage around the breast and/or one or more slits so a “cookie” can be inserted, where the cookie can provide extra breast tissue support and shape and optionally extra coverage around the nipple area of the breast. The cookie may comprise a single textile layer or may comprise multiple textile layers optionally with padding contained between layers. While the liner is only shown for an upper torso garment, the liner can be included in each of the garments illustrated in FIGS. 23A to 26B as well as in other similar garments covered by the present disclosure.

As described herein the phrase “upper torso” includes the thoracic segment of the torso, the phrase “half torso” refers to the thoracic segment and the abdominal segment of the torso, and the phrase “full torso” includes the thoracic segment, the abdominal segment, and the lower torso (also referred to as the pelvic and perineal segments).

The upper torso garment can be sized and shaped to be worn over the following major muscle groups: the pectoralis major, the upper rectus abdominis, and the upper external oblique muscle groups (on the front side of the body) and upper latissimus dorsi and lower trapezius muscle groups (on the back side of the body). The upper torso garment can also include one or more straps (which are not part of the single engineered warp knitted component) that can extend from the front to the back and are worn over a portion the trapezius muscle group. The upper torso garment can include one or more straps, such as when the upper torso garment is a bra, a tank top, a camisole, or a bodysuit. In one aspect, only a single strap (i.e., a right side strap or a left side strap) can be included. Alternatively, the upper torso garment can include both a right side strap and a left side strap. The strap or straps of the upper torso garment can be narrow, such as less than 1 cm in width, or may be wider, such as 1 cm in width or greater than 1 cm in width. When the upper torso garment is a shirt or a bodysuit, it can include a yoke sized and shaped to cover a right side of the chest and right shoulder and a left side of the chest and left shoulder. Optionally, the upper torso garment can include a right sleeve attached to the right side of the yoke and a left sleeve attached to the left side of the yoke. The sleeves can be long sleeves, elbow length sleeves, or short sleeves.

The half torso garment can be sized and shaped to be worn over the muscle groups described in reference to the upper torso garment as well as the following major muscle groups: the rectus abdominis and external oblique muscle groups (on the front side of the body) and latissimus dorsi (on the back side of the body). The half torso garment can also include one or more straps that can extend from the front to the back and are worn over a portion the trapezius muscle group, or can include a yoke that extends from the front to the back on both the left and right sides, and is worn over a portion of the left and right trapezius muscle groups. As with the upper torso garment, the straps can be narrow or wide. Optionally, when the half torso garment includes a yoke, the half torso garment can also include one or more sleeves attached to the yoke.

The full torso garment can be sized and shaped to be worn over the muscle groups described in reference to the upper torso garment and half torso garment as well as following major muscle groups: the lower rectus abdominis that extend into the pelvic area (on the front side of the body) and upper gluteus maximus and upper hip muscle groups (on the back side and hip area of the body). The full torso garment can also include one or more straps or a yoke that can extend from the front to the back and are sized and shaped to be worn over a portion the trapezius muscle group. As with the upper torso garment and the half torso garment, the one or more straps can be narrow or wide, and one or more sleeves can be attached to the yoke. For embodiments where the full torso garment includes a thong-cut bottom portion, the thong can extend from the back of the body (lower back) to the front of the body and may be sized and shaped to cover a portion of the gluteus maximus, perineal, and pelvic areas. Further, an embodiment of the full torso garment can be sized and shaped to include full coverage of the gluteus maximus, perincal, and pelvic areas. In another embodiment, the full torso garment can include a bottom portion including shorts sized and shaped to cover an upper area of the thigh, the upper portion of the quadriceps and hamstring muscle groups and can optionally cover the mid to lower areas of the thigh to just above (e.g., about 2 inches, about 1 inch, about 0.5 inch) the knee joint. In some embodiments, a length of the inseam of the shorts portion can be about 2 inches, about 4 inches, about 6 inches, etc.

In regard to the discussion regarding the muscle groups, the recited muscle groups do not represent all of the muscle groups that are covered by the garment, but the description is intended to provide the main muscle groups and to provide the context of the areas of the body that a garment is sized and shaped to cover in conjunction with reference to the upper, mid-, and lower torso areas.

Having described aspects of the present disclosure generally in regard to FIGS. 23A and 23B, FIGS. 23A and 23B will now be described in additional detail. FIGS. 23A and 23B illustrate an upper torso garment (3000). The upper torso garment (3000) includes a front side (FIG. 23A) and a rear side (FIG. 23B). The front side is sized and shaped to cover an area corresponding to a front portion of a wearer's upper torso and the rear side is sized and shaped to cover an area corresponding to a rear portion of a wearer's upper torso. The front and rear sides comprise a single engineered warp knitted component (3001, 4001, 5001, 6001, 8001a, 8001b, 9001) having a plurality of integrated compression zones, where multiple compression zones have different amounts of compression (e.g., amounts of compression modulus) and fabric weight. In an aspect, two or more compression zones can have the same amounts of compression and fabric weight.

The single engineered warp knitted component (3001) of the garment (3000) has a first compression zone on the front side, where the first compression zone has a first amount of compression (e.g., compression modulus) and a first fabric weight. The first compression zone includes a first right compression zone (3002r) and a first left compression zone (3002l). The first right compression zone (3002r) includes a region sized and shaped to cover at least a portion of an area corresponding to a wearer's right breast, while the first left compression zone (3002l) includes a region sized and shaped to cover at least a portion of an area corresponding to a wearer's left breast. The first right compression zone (3002r) and the first left compression zone (3002l) can be sized based on the cup sizes (e.g., AA to H) for a bra. In general, the first amount of compression and the first fabric weight are less than the amounts of compression and fabric weight of other areas of the upper torso garment (3000).

The single engineered warp knitted component (3001) of the garment (3000) includes a second compression zone on the front side of the garment. The second compression zone has a second amount of compression and a second fabric weight. The second compression zone includes a second right compression zone (3004r), a second left compression zone (3004l), and a second middle compression zone (3004m) between the second right compression zone (3004r) and the second left compression zone (3004l), The second right compression zone (3004r) includes a region sized and shaped to cover an area corresponding to a portion of a wearer's torso around the root of the breast. The second right compression zone (3004r) extends in width at least along a bottom edge of the first right compression zone (3002r). The second left compression zone (3004l) includes a region sized and shaped to cover an area corresponding to a portion of a wearer's torso around the root of the breast. The second left compression zone (3004l) extends at least along a bottom edge of the first left compression zone (3002l). The second middle compression zone (3004m) includes a region sized and shaped to cover an area between the right breast area and the left breast area and may connect the second left compression zone (3004l) and the second right compression zone (3004r) as illustrated in FIG. 23A. As illustrated in FIG. 23A, the second right compression zone (3004r), the second middle compression zone (3004m), and the second left compression zone (3004l) form a single continuous zone across a majority of the width of the front side of the garment. Alternatively, the garment (3000) can include only the second right compression zone (3004r) and the second left compression zone (3004l), and the second right compression zone (3004r) and the second left compression zone (3004l) are not connected to each other.

The single engineered warp knitted component (3001) of the garment (3000) may include a third compression zone (3006) on the front side of the garment. The third compression zone (3006) has a third amount of compression and a third fabric weight. As illustrated in FIG. 23A, the third compression zone (3006) is sized and shaped to cover at least a portion of an area corresponding to a wearer's front torso below both the right and left breast areas and between the right and left breast areas. Also as illustrated in FIG. 23A, the third compression zone (3006) is below the second middle compression zone (3004m) and below portions of both the first left compression zone (3002l) and the first right compression zone (3002r) and extends in width at least between a portion of the bottom of the second right compression zone (3004r) and a portion of the bottom of the second left compression zone (3004l). Alternatively, the third compression zone (3006) can be sized and shaped to cover at least a portion of an area of a wearer's front torso between the right and left breast areas, or can be a third right compression zone sized and shaped to cover an area below at least a portion of the right breast area, and a separate third left compression zone sized and shaped to cover an area below at least a portion of the left breast area.

In regard to how the various zones are connected in the single engineered warp knitted component (3001), the following provides embodiments on how the various zones can be connected. In an aspect, the first right compression zone (3002r) is directly or indirectly joined to the second right compression zone (3004r) by a first seamless transition with the first right compression zone (3002r). In an aspect, the first left compression zone (3002l) is directly or indirectly joined to the second left compression zone (3004l) by a second seamless transition with the first left compression zone (3002l). In an aspect, the first left compression zone (3002l) and the first right compression zone (3002r) are directly or indirectly joined to the second middle compression zone (3004m) by a second seamless transition with the second middle compression zone (3004m). In an aspect, the second right compression zone (3004r), the second left compression zone (3004l), and second middle compression zone (3004m) are directly or indirectly joined to the third compression zone (3006) by a third seamless transition with the third compression zone (3006).

In regard to the amounts of compression for the first, second, and third zones, the first amount of compression is less than the second amount of compression, and the first amount of compression is less than the third amount of compression. As illustrated in FIGS. 23A and 23B, the second amount of compression is greater than the third amount of compression. Compression zones 3002r and 3002l have the first amount of compression, compression zones 3004r, 3004l and 3004m have the second amount of compression which is greater than the first amount of compression, and compression zone 3006 has the third amount of compression which is greater than the first amount of compression but less than the second amount of compression. Alternative embodiments may have different relationships between the amounts of compression of the various compression zones.

In regard to the fabric weight for the first, second, and third zones, as illustrated in FIGS. 23A and 23B, the first fabric weight is less than the second fabric weight and is less than the third fabric weight, and the second fabric weight is greater than the third fabric weight. Compression zones 3002r and 3002l have the first fabric weight, compression zones 3004r, 3004l, and 3004m have the second fabric weight which is greater than the first fabric weight, and compression zone 3006 has the third fabric weight which is greater than the first fabric weight but less than the second fabric weight. In one aspect, a difference in fabric weight between the first fabric weight and the second fabric weight or between the first fabric weight and the third fabric weight ranges from 100 GSM to 220 GSM. In some embodiments, a difference in fabric weight between the first fabric weight and the second fabric weight or between the first fabric weight and the third fabric weight ranges from 20 GSM to 220 GSM. Alternative embodiments may have different relationships between the fabric weights of the various compression zones.

In regard to the first, second, and third compression zones, at least one of a) the first right compression zone (3002r) and the first left compression zone (3002l), b) the second right compression zone (3004r) and the second left compression zone (3004l), and c) the third compression zone (3006) of the single engineered warp knitted component (3001) comprises a jacquard knit stitch construction. In an aspect, two of the first, second and third zones comprise a jacquard knit stitch construction. In another aspect, all three of the first, second, and third compression zones comprise a jacquard knit stitch construction. With regard to FIG. 23A, in one aspect, compression zones 3002r and 3002l comprise a first jacquard knit stitch construction. In another aspect, the combined compression zones 3004r, 3004l, and 3004m comprise a second jacquard knit stitch construction. In another aspect, compression zone 3006 comprises a third jacquard knit stitch construction. In yet another aspect, the combined compression zones 3004r, 3004l, and 3004m comprise a second jacquard knit stitch construction, and compression zone 3006 comprises a third jacquard knit stitch construction, optionally compression zones 3002r and 3002l may comprise a first jacquard knit stitch construction. Alternative embodiments may include different distributions of jacquard knit stitch constructions among the various compression zones.

The jacquard knit stitch construction includes an elastane jacquard knit stitch construction, and the elastane jacquard knit stitch construction is configured to increase a density or configured to increase a fabric weight or configured to increase both a density and a fabric weight in the portion of the garment (3000) in which the jacquard knit stitch construction is present as compared to a portion of the garment (3000) without the jacquard knit stitch construction. In one embodiment, the jacquard knit stitch construction and/or the elastane jacquard knit stitched construction can be independently knitted and can be seamlessly added to the first (ground) knit construction using the second jacquard bar 920. This construction technique allows a single engineered warp knitted component (3001) to be engineered to include a plurality of integrated compression zones such that the size, shape, compression, fabric weight or any combination thereof may be varied. For example, using this construction technique, the size, shape and compression moduli of the first right compression zone and the first left compression zone; the second right compression zone, the second left compression zone and the second middle compression zone; and the third compression zone can be engineered by changing the size and/or shape of the zone, the size (denier/filament numbers) and/or cross-sectional shape of the yarn, yarn combination and the jacquard knit stitch construction may be varied across the compression zones in a single engineered warp knitted component (3001) included in a garment (3000).

The single engineered warp knitted component (3001) of the garment (3000) can also include a fourth compression zone having a fourth amount of compression and a fourth fabric weight. In the embodiment illustrated in FIGS. 23A, the fourth compression zone includes a fourth right compression zone (3008r) and a fourth left compression zone (3008l). The fourth right compression zone is sized and shaped to cover at least a portion of an area corresponding to a wearer's front upper torso below at least a portion of the right breast area and extends past the right breast area along the side upper torso. The fourth right compression zone (3008r) is below both the first right compression zone (3002r) and the second right compression zone (3004r). The fourth right compression zone (3008r) extends in width at least along the bottom and side of the second right compression zone (3004r) and, as illustrated in FIG. 23B, may extend from a right front side to a right back side (3008lb). The fourth left compression zone (3008l) is sized and shaped to cover at least a portion of an area corresponding to a wearer's front upper torso below at least a portion of the left breast area and extends past the left breast area along the side upper torso. The fourth left compression zone (3008l) is below both the first left compression zone (3002l) and the second left compression zone (3004l). The fourth left compression zone (3008l) extends in width at least along the bottom and side of the second left compression zone (3004l) and, as illustrated in FIG. 23B, may extend from a left front side to a left back side (3008rb). In another embodiment, the fourth compression zone can be a single zone including a fourth right compression zone, a fourth left compression zone, and a fourth middle compression zone between the fourth right compression zone and the fourth left compression zone. In another embodiment, the fourth compression zones (3008r and 3008l) are present only on a front of the garment or on the portion of the single engineered warp knitted component (3001) configured to form a front of the garment. In another embodiment, the garment or single engineered warp knitted component (3001) of the garment includes one or more fourth compression zones in which the one or more fourth compression zones are positioned differently than as illustrated in FIG. 23A. In yet another embodiment, the garment or single engineered warp knitted component (3001) does not include a fourth compression zone having a different level of compression, a different fabric weight, a different jacquard knit stitch construction, or any combination thereof, as compared the first, second and third compression zones.

In regard to how the various zones of the single engineered warp knitted component (3001) are connected, the following provides embodiments on how the various zones can be connected. As illustrated in FIG. 23A, the fourth right compression zone (3008r) is directly or indirectly joined with the second right compression zone (3004r) by a seamless transition and is directly or indirectly joined with the third compression zone (3006) by a seamless transition. The fourth left compression zone (3008l) is directly or indirectly joined with the second left compression zone (3004l) by a seamless transition and is directly or indirectly joined with the third compression zone (3006) by a seamless transition. The fourth right compression zone (3008r) and fourth left compression zone (3008l) can comprise jacquard knit stitch construction and can include an elastane jacquard knit stitch construction. The elastane jacquard knit stitch construction is configured to engineer a density or a fabric weight in the fourth right and left compression zones (3008r and 3008l). The jacquard knit stitch construction and/or the elastane jacquard knit stitched construction can be independently constructed using the second jacquard guide bar 920 and seamlessly added to the first (ground) knit construction constructed using the first jacquard guide bar 910.

In regard to the amount of compression of the fourth compression zone, as illustrated in FIG. 23A, the fourth right compression zone (3008r) and the fourth left compression zone (3008l) both have the fourth amount of compression. In embodiments where the side compression zones extend from the front of the garment to the back of the garment as illustrated in FIG. 23B, the fourth right compression zone (3008r) extends into the fourth right back compression zone (3008lb), the fourth left compression zone (3008l) extends into the fourth left back compression zone (3008rb), and each have the fourth amount of compression. The fourth amount compression is greater than the first amount of compression, the fourth amount of compression is greater than the second amount of compression, and the fourth amount of compression is greater than the third amount of compression. Alternative embodiments may have different relationships between the amounts of compression of the various compression zones.

In regard to the fabric weight for the first and fourth zones, as illustrated in FIG. 23A, the fourth right compression zone (3008r) and the fourth left compression zone (3008l) both have the fourth fabric weight, and the fourth fabric weight is greater than the first fabric weight, the fourth fabric weight is greater than the second fabric weight, and the fourth fabric weight is greater than the third fabric weight. In one aspect, a difference between the fourth fabric weight and the first fabric weight ranges from 20 GSM to 220 GSM. Alternative embodiments may have different relationships between the fabric weights of the various compression zones.

In the embodiment illustrated in FIG. 23A, the single engineered warp knitted component (3001) of the garment (3000) also includes a fifth compression zone (3012f) on the front side having a fifth amount of compression and a fifth fabric weight. The fifth compression zone (3012f) is below at least a portion of the first right compression zone (3002r), at least a portion of the first left compression zone (3002l), at least a portion of the second right compression zone (3004r), at least a portion of the second left compression zone (3004l), and at least a portion of the third compression zone (3006) and extends laterally at least between the fourth right compression zone (3008r) and the fourth left compression zone (3008l). In another embodiment, when the fourth compression zone includes the fourth right compression zone (3008r) and the fourth left compression zone (3008l), the area occupied by the fifth compression zone on the front side may be positioned between the fourth right compression zone and the fourth left compression zone on the side of the single engineered warp knitted component (3001) configured to form the front of the garment (3000). In another embodiment, the fifth compression zone (3012f) is present only on a side of the single engineered warp knitted component (3001) configured to form a front side of the garment, or is present only on portion of the single engineered warp knitted component (3001) configured to form a back side of the garment. In another embodiment, the garment or single engineered warp knitted component (3001) of the garment may include a fifth compression zone in which the fifth compression zone is positioned differently than as illustrated in FIG. 23A. In yet another embodiment, the garment or single engineered warp knitted component (3001) does not include a fifth compression zone having a different level of compression, a different fabric weight, a different jacquard knit stitch construction, or any combination thereof, as compared the first, second, third and fourth compression zones.

In an aspect, the fifth compression zone (3012f) is directly or indirectly joined with the third compression zone (3006) by a seamless transition. The fifth compression zone (3012f) can be directly or indirectly joined with the fourth left compression zone (3008l) by a seamless transition and with the fourth right compression zone (3008r) by a seamless transition.

In regard to the amount of compression of the fifth compression zone, as illustrated in FIG. 23A, the fifth amount of compression of the fifth compression zone (3012f) is greater than the first amount of compression, the fifth amount of compression is greater than the second amount of compression, the fifth amount of compression is greater than the third amount of compression, and the fifth amount of compression is greater than the fourth amount of compression. Alternative embodiments may have different relationships between the amounts of compression of the various compression zones.

In regard to the fabric weight for the first and fifth compression zones (3002r/l and 3012f) as illustrated in FIG. 23A, the first fabric weight is less than the fifth fabric weight. In an aspect, a difference between the fifth fabric weight and the first fabric weight can range from 20 GSM to 220 GSM. Alternative embodiments may have different relationships between the fabric weights of the various compression zones.

In the embodiment illustrated in FIG. 23B, the single engineered warp knitted component (3001) of garment (3000) also has a fifth back compression zone (3012b) on the back side of the garment having the fifth amount of compression and the fifth fabric weight. The fifth back compression zone (3012b) is between the right back side (3008lb) of the fourth compression zone and the left back side (3008rb) of the fourth compression zone. The fifth back compression zone (3012b) is directly or indirectly joined with the right back side (3008lb) of the fourth compression zone and the left back side (3008rb) of the fourth compression zone by a seamless transition. In an alternative embodiment, the back side of the garment can include at least one back compression zone located between the back right side and the back left side of the garment, and the at least one back compression zone can have an amount of compression greater than the first compression zone, or a fabric weight greater than the first fabric weight. In another alternative embodiment, the entire back side of the garment includes a single compression zone, and the single compression zone can have an amount of compression greater than the first compression zone, or a fabric weight greater than the first fabric weight.

In the embodiment illustrated in FIG. 23A the garment (3000) also includes a right side strap (3024r) and a left side strap (30241) (both of which are not part of the single engineered warp knitted component 3001), where each of the right side strap and the left side strap extend from the front side to the back side of garment (3000). As illustrated in FIGS. 23A and 23B, the single engineered warp knitted component (3001) of the garment (3000) forms a front portion of each of the right side strap (3024r) and the left side strap (30241). In an aspect, each of the right side strap (3024r) and the left side strap (30241) extends from the front side to the back side, but as illustrated in FIGS. 23A and 23B, the portions of the single engineered warp knitted component (3001) forming the left and right straps do not extend from the front side of the garment (3000) to the back side of the garment (3000). As illustrated in FIG. 23B, ends of the portions forming the left and right straps are affixed to separate strap components (30441 and 3044r, respectively) configured to allow the straps to be adjusted in length. As illustrated in FIG. 23B, separate strap components (30441 and 3044r) are not part of the single engineered warp knitted component (3001) of the garment (3000). In alternative embodiments, all of the right and left straps (3024r and 3024l) may be part of the single engineered warp knitted component (3001) such that the full length of the straps are integrally knitted as part of the single engineered warp knitted component (3001), and adjustment hardware may be added to the strap portions before ends of the straps are directly affixed to an opposite side of the single engineered warp knitted component (3001). As shown in FIG. 23B, the opposite ends of the left and right strap components (30441 and 3044r) are affixed to the back top compression zone 3016. In other embodiments, the opposite ends to the left and right strap components (30441 and 3044r) may be affixed to any compression zone on the back side of the garment (3000), such as, for example, the opposite end of the right strap component (3044r) may be affixed to the left back side 3008rb of the fourth compression zone, and the opposite end of the left strap component (30441) may be affixed to the right back side compression zone 3008lb of the fourth compression zone. In the aspect illustrated in FIGS. 23A and 23B, when the single engineered warp knitted component (3001) includes a portion of the right side strap (3024r), the portion of the right side strap (3024r) comprises a sixth right compression zone (3014r) and the portion of the left side strap (30241) comprises a sixth left compression zone (30141) equal to the second amount of compression. The fabric weight of the sixth fabric zone is the same as the fabric weight of the second compression zones. In alternative embodiments, the right side strap (3024r) and the left side strap (30241) can have an amount of compression that is greater than the fifth amount of compression, or can be equal to or less than to the fifth amount of compression. The right side strap (3024r) and the left side strap (30241) can have an amount of compression that is greater than the fourth amount of compression, or can be equal to or less than to the fourth amount of compression. The right side strap (3024r) and the left side strap (30241) can have an amount of compression that is greater than the third amount of compression, or can be equal to or less than to the third amount of compression. The right side strap (3024r) and the left side strap (30241) can have an amount of compression that is greater than the second amount of compression, or can be equal to or less than to the second amount of compression. The right side strap (3024r) and the left side strap (30241) can have an amount of compression that is greater than the first amount of compression, or can be equal to or less than to the first amount of compression. In these alternative embodiments, the right side strap (3024r) and the left side strap (30241) can have a fabric weight that is greater than the fifth fabric weight, or can be equal to or less than to the fifth fabric weight. The right side strap (3024r) and the left side strap (30241) can have a fabric weight that is greater than the fabric weight, or can be equal to or less than to the fourth fabric weight. The right side strap (3024r) and the left side strap (30241) can have a fabric weight that is greater than the third fabric weight, or can be equal to or less than to the third fabric weight. The right side strap (3024r) and the left side strap (30241) can have a fabric weight that is greater than the second fabric weight, or can be equal to or less than to the second fabric weight. The right side strap (3024r) and the left side strap (30241) can have a fabric weight that is greater than the first fabric weight, or can be equal to or less than the first fabric weight.

In the embodiment illustrated in FIG. 23A, the single engineered warp knitted component (3001) of garment (3000) includes the sixth compression zone having the second amount of compression and the second fabric weight. The sixth compression zone includes a sixth right compression zone (3014r) and a sixth left compression zone (30141). The sixth right compression zone (3014r) is sized and shaped to cover at least a portion of an area corresponding to a wearer's front shoulder above the right breast area. The sixth right compression zone (3014r) is above the first right compression zone (3002r). The sixth left compression zone (30141) is sized and shaped to cover at least a portion of an area corresponding to a wearer's front shoulder above the left breast area. The sixth left compression zone (30141) is above the first left compression zone (3002l). In an aspect, the sixth right compression zone (3014r) is directly or indirectly joined with the first right compression zone (3002r) by a seamless transition. The sixth left compression zone (30141) is directly or indirectly joined with the first left compression zone (3002l) by a seamless transition.

In the embodiment illustrated in FIG. 23B, the single engineered warp knitted component (3001) of garment (3000) includes a back top compression zone (3016) on the back side having the third amount of compression and the third fabric weight. The back top compression zone (3016) is above and extends between the right back side (3008lb) of the fourth compression zone and the left back side (3008rb) of the fourth compression zone and above the fifth back compression zone (3012b). The back top compression zone (3016) is directly or indirectly joined with the right back side (3008lb) of the fourth compression zone and the left back side (3008rb) of the fourth compression zone, and also directly or indirectly joined with the fifth back compression zone (3012b) by a seamless transition. In an alternative embodiment, the garment can include a back top compression zone (3016) having the first amount of compression, the second amount of compression, the fourth amount of compression, or the fifth amount of compression, or an amount of compression different than any of the other compression zones; or the back top compression zone (3016) can have the first fabric weight, the second fabric weight, the fourth fabric weight, the fifth fabric weight, or a fabric weight different than any of the other compression zones. In another alternative embodiment, the back of the garment includes a single back compression zone, and the single back compression zone includes the back top region.

In an aspect, one or more of the first compression zone, the second compression zone, the third compression zone, the fourth compression zone, the fifth compression zone, the sixth compression zone, and the back top compression zone of the single engineered warp knitted component (3001) include the jacquard knit stitch construction. In an aspect, two or more of the first compression zone, the second compression zone, the third compression zone, the fourth compression zone, the fifth compression zone, the sixth compression zone, and the back top compression zone include the jacquard knit stitch construction. In an aspect, three or more of the first compression zone, the second compression zone, the third compression zone, the fourth compression zone, the fifth compression zone, the sixth compression zone, and the back top compression zone include the jacquard knit stitch construction. In an aspect, four or more of the first compression zone, the second compression zone, the third compression zone, the fourth compression zone, the fifth compression zone, the sixth compression zone, and the back top compression zone include the jacquard knit stitch construction. Each of the first compression zone, the second compression zone, the third compression zone, the fourth compression zone, the fifth compression zone, the sixth compression zone, and the back top compression zone each include the jacquard knit stitch construction.

In regard to the amounts of compression, when four different levels of compression are present in the single engineered warp knitted component (3001, 4001, 5001, 6001, 8001a, 8001b, 9001) of a garment (3000, 4000, 5000, 6000, 8000a, 8000b, and 9000), the order of the amount of compression from lowest to highest amount of compression can be: first amount, third amount, second amount, and fourth amount. In alternative embodiment, the order of the amount compression from lowest to highest amount of compression can be: first amount, third amount, fourth amount, and second amount. In another embodiment, the order of the amount of compression from lowest to highest amount of compression can be: first amount, fourth amount, third amount, and second amount.

In an aspect, when four different fabric weights are present in the single engineered warp knitted component (3001, 4001, 5001, 6001, 8001a, 8001b, 9001) of a garment (3000, 4000, 5000, 6000, 8000a, 8000b, and 9000), the order of the fabric weight from lowest to highest can be as follows: a) first fabric weight, third fabric weight, second fabric weight, fourth fabric weight; or b) first fabric weight, third fabric weight, fourth fabric weight, and second fabric weight; or c) first fabric weight, fourth fabric weight, third fabric weight, and second fabric weight.

In regard to the amounts of compression, when five different levels of compression are present in the single engineered warp knitted component (3001, 4001, 5001, 6001, 8001a, 8001b, 9001) of a garment (3000, 4000, 5000, 6000, 8000a, 8000b, and 9000), the order of the amount of compression from lowest to highest amount of compression can be: first amount, third amount, second amount, fourth amount, and fifth amount. In an alternative embodiment, the order of the amount of compression from lowest to highest amount of compression can be: first amount, third amount, fourth amount, second amount, and fifth amount. In another embodiment, the order of the amount of compression from lowest to highest amount of compression can be: first amount, fourth amount, third amount, second amount, and fifth amount.

In an aspect, when five different fabric weights are present in the single engineered warp knitted component (3001, 4001, 5001, 6001, 8001a, 8001b, 9001) of a garment (3000, 4000, 5000, 6000, 8000a, 8000b, and 9000), the order of the fabric weight from lowest to highest can be as follows: a) first fabric weight, third fabric weight, second fabric weight, fourth fabric weight, and fifth fabric weight; or b) first fabric weight, third fabric weight, fourth fabric weight, second fabric weight and fifth fabric weight; or c) first fabric weight, fourth fabric weight, third fabric weight, second fabric weight, and fifth fabric weight.

In regard to the amounts of compression, when seven different levels of compression are present in the single engineered warp knitted component (3001, 4001, 5001, 6001, 8001a, 8001b, 9001) of a garment (3000, 4000, 5000, 6000, 8000a, 8000b, and 9000), the order of the amount of compression from lowest to highest amount of compression can be: first amount, third amount, second/sixth amount, fourth amount, seventh amount, and fifth amount. In alternative embodiment, the order of the amount of compression from lowest to highest amount of compression can be: first amount, third amount, fourth amount, second/sixth amount, seventh amount, and fifth amount. In another embodiment, the order of the amount of compression from lowest to highest amount of compression can be: first amount, fourth amount, third amount, second/sixth amount, seventh amount, and fifth amount. In an embodiment the second amount and the sixth amount are the same, while in another embodiment the second amount can be greater than the sixth amount or the sixth amount can be greater than the second amount.

In an aspect, when seven different fabric weights are present in the single engineered warp knitted component (3001, 4001, 5001, 6001, 8001a, 8001b, 9001) of a garment (3000, 4000, 5000, 6000, 8000a, 8000b, and 9000), the order of the fabric weight from lowest to highest can be as follows: a) first fabric weight, third fabric weight, second/sixth fabric weight, fourth fabric weight, seventh fabric weight, and fifth fabric weight; or b) first fabric weight, third fabric weight, fourth fabric weight, second/sixth fabric weight, seventh fabric weight, and fifth fabric weight; or c) first fabric weight, fourth fabric weight, third fabric weight, second/sixth fabric weight, seventh fabric weight, and fifth fabric weight.

FIGS. 24A and 24B illustrate a half torso garment (4000), which is designed to cover the upper torso and the mid-torso areas. The single engineered warp knitted component (4001) of half torso garment (4000) and half torso garment (4000) are similar to and includes the elements described with respect to the single engineered warp knitted component (3001) of upper torso garment (3000), but the single engineered warp knitted component of half torso garment (4000) and half torso garment (4000) are longer to cover the mid-torso. As a result, the fourth compression zone (a fourth right compression zone (4008r) and a fourth left compression zone (40081)) and the fifth compression zone (4012f and 4012b) are longer than the corresponding zones in the upper torso garment (3000) and in the single engineered warp knitted component (3001) of upper torso garment (3000).

The upper portion (e.g., bra portion) of the half torso garment (4000) and the single engineered warp knitted component (4001) of half torso garment (4000) are similar to or the same as those for the garment (3000). In this regard, the description and discussion regarding single engineered warp knitted component (3001) of upper torso garment (3000), including the first right compression zone (3002r), the first left compression zone (3002l), the second right compression zone (3004r), the second left compression zone (3004l), the second middle compression zone (3004m), the third compression zone (3006), the sixth right compression zone (3014r), the sixth left compression zone (30141), the right side strap (3024r), the left side strap (30241), and the back top compression zone (3016) apply to the single engineered warp knitted component (4001) of half torso garment (4000) and of half torso garment (4000) and are not repeated in the discussion regarding the single engineered warp knitted component (4001) of half torso garment (4000) and half torso garment (4000). The following discussion regarding the single engineered warp knitted component (4001) of half torso garment (4000) and to half torso garment (4000) are directed to additional features of the single engineered warp knitted component (4001) of half torso garment (4000) and the half torso garment (4000).

The half torso garment (4000) and the single engineered warp knitted component (4001) of half torso garment (4000) include a front side (FIG. 24A) and a rear side (FIG. 24B). The front side is sized and shaped to cover an area corresponding to a front portion of a wearer's upper and mid-torso and the rear side is sized and shaped to cover an area corresponding to a rear portion of a wearer's upper and mid-torso. The front and rear sides of half torso garment (4000) comprise a single engineered warp knitted component (4001) having a plurality of integrated compression zones, where multiple compression zones have different amounts of compression and fabric weight. In an aspect, two or more compression zones can have the same amounts of compression and fabric weight.

In the embodiment illustrated in FIGS. 24A and 24B, the single engineered warp knitted component (4001) of garment (4000) also includes a fourth compression zone having a fourth amount of compression and a fourth fabric weight. The fourth compression zone includes a fourth right compression zone (4008r) and a fourth left compression zone (40081). The fourth right compression zone (4008r) is sized and shaped to cover at least a portion of an area corresponding to a wearer's front and mid-torso below the right breast area and the side area. The fourth right compression zone (4008r) is below both the first right compression zone (3002r) and the second right compression zone (3004r). The fourth right compression zone (4008r) extends in width at least along the side of the second right compression zone (3004r) and extends from a right front side to a right back side (40081b). The fourth left compression zone (40081) is sized and shaped to cover at least a portion of an area corresponding to a wearer's front and mid-torso below the left breast area and side area. The fourth left compression zone (40081) is below both the first left compression zone (3002l) and the second left compression zone (3004l). The fourth left compression zone (40081) extends in width at least along the side of the second left compression zone (3004l) and extends from a left front side to a left back side (4008rb). In another embodiment, the fourth compression zone of the single engineered warp knitted component (4001) can be a single zone including a fourth right compression zone, a fourth left compression zone, and a fourth middle compression zone between the fourth right compression zone and the fourth left compression zone. In another embodiment, the fourth compression zones (4008r and 40081) are present only on a front of the garment or on the portion of the single engineered warp knitted component (4001) configured to form a front of the garment. In another embodiment, the garment (4000) or single engineered warp knitted component (4001) of the garment includes one or more fourth compression zones in which the one or more fourth compression zones are positioned differently than as illustrated in FIG. 23A or 23B. In yet another embodiment, the garment (4000) or single engineered warp knitted component (4001) does not include a fourth compression zone having a different level of compression, a different fabric weight, a different jacquard knit stitch construction, or any combination thereof, as compared to the first, second and third compression zones.

In regard to how the various zones are connected in FIGS. 24A and 24B, the following provides embodiments on how the various zones can be connected in the single engineered warp knitted component (4001). The fourth right compression zone (4008r) is directly or indirectly joined with the second right compression zone (3004r) by a seamless transition and is directly or indirectly joined with the third compression zone (3006) by a seamless transition. The fourth left compression zone (40081) is directly or indirectly joined with the second left compression zone (3004l) by a seamless transition and is directly or indirectly joined with the third compression zone (3006) by a seamless transition. The fourth right compression zone (4008r) and left compression zone (40081) can comprise jacquard knit stitch construction and can include an elastane jacquard knit stitch construction. The elastane jacquard knit stitch construction is configured to engineer a density or a fabric weight in the fourth right compression zone (4008r) and left compression zone (40081). The jacquard knit stitch construction and/or the elastane jacquard knit stitched construction can be independently knitted and can be seamlessly added to the first (ground) knit construction using the second jacquard bar (920).

In regard to the amount of compression of the fourth compression zone, as illustrated in FIG. 24A, the fourth right compression zone (4008r) and the fourth left compression zone (40081) both have the fourth amount of compression. In embodiments where the side compression zones extend from the front of the garment to the back of the garment as illustrated in FIG. 24B, the fourth right compression zone (4008r) extends into the fourth right back compression zone (40081b), the fourth left compression zone (40081) extends into the fourth left back compression zone (4008rb), and each have the fourth amount of compression. The fourth amount compression is greater than the first amount of compression, the fourth amount of compression is greater than the second amount of compression, and the fourth amount of compression is greater than the third amount of compression. Alternative embodiments may have different relationships between the amounts of compression of the various compression zones.

In regard to the fabric weight for the first and fourth zones, as illustrated in FIG. 24A, the fourth right compression zone (4008r) and the fourth left compression zone (40081) both have the fourth fabric weight, and the fourth fabric weight is greater than the first fabric weight, the fourth fabric weight is greater than the second fabric weight, and the fourth fabric weight is greater than the third fabric weight. In one aspect, a difference between the fourth fabric weight and the first fabric weight ranges from 20 GSM to 220 GSM. Alternative embodiments may have different relationships between the fabric weights of the various compression zones.

In the embodiment illustrated in FIGS. 24A and 24B, the single engineered warp knitted component (4001) of garment (4000) also includes a fifth compression zone (4012f) on the front side having a fifth amount of compression and a fifth fabric weight. The fifth compression zone (4012f) is sized and shaped to cover the upper and mid-torso. The fifth compression zone (4012f) is below at least a portion of the first right compression zone (3002r), at least a portion of the first left compression zone (3002l), at least a portion of the second right compression zone (3004r), at least a portion of the second left compression zone (3004l), and at least a portion of the third compression zone (3006) and extends laterally at least between the fourth right compression zone (4008r) and the fourth left compression zone (40081). In another embodiment, the fourth compression zone can include the fourth right compression zone (4008r), the fourth left compression zone (40081), and the area occupied by the fifth compression zone (4012f) on the front side as described above, in which the entire fourth compression zone has the fourth amount of compression and the fourth fabric weight. Optionally, the fourth right compression zone (4008r) and the fourth left compression zone (40081) can each individually extend from a front side of the garment to a back side of the garment. In another embodiment, the single engineered warp knitted component (4001) of the garment includes only a fifth front compression zone present on a side of the single engineered warp knitted component (4001) configured to form a front side of the garment, or includes the fifth back compression zone present only on a side of the engineered warp knitted component (4001) configured to form a back side of the garment. In another embodiment, the garment or single engineered warp knitted component (4001) of the garment includes a fifth compression zone in which the fifth compression zone is positioned differently than as illustrated in FIG. 24A or 24B. In yet another embodiment, the garment or single engineered warp knitted component (4001) does not include a fifth compression zone having a different level of compression, a different fabric weight, a different jacquard knit stitch construction, or any combination thereof, as compared to the first, second, third and fourth compression zones.

In the embodiment illustrated in FIGS. 24A and 24B, the fifth compression zone (4012f) is directly or indirectly joined with the third compression zone (3006) by a seamless transition. The fifth compression zone (4012f) can be directly or indirectly joined with the fourth left compression zone (40081) by a seamless transition and the fourth right compression zone (4008r) by a seamless transition in the single engineered warp knitted component (4001).

In regard to the amount of compression of the fifth compression zone (4012f), as illustrated in FIGS. 24A and 24B, the fifth amount of compression of the fifth compression zone is greater than the first amount of compression, the fifth amount of compression is greater than the second amount of compression, the fifth amount of compression is greater than the third amount of compression, and the fifth amount of compression is greater than the fourth amount of compression. Alternative embodiments may have different relationships between the amounts of compression of the various compression zones.

In regard to the fabric weight for the first compression zone (3002r and 3002l) and fifth compression zone (4012f) as illustrated in FIGS. 24A and 24B, the first fabric weight is less than the fifth fabric weight. In an aspect, a difference between the fifth fabric weight and the first fabric weight can range from 20 GSM to 220 GSM. Alternative embodiments may have different relationships between the fabric weights of the various compression zones.

In the embodiment illustrated in FIGS. 24A and 24B, the single engineered warp knitted component (4001) of garment (4000) also has a fifth back compression zone (4012b) on the back side having the fifth amount of compression and the fifth fabric weight. The fifth back compression zone (4012b) covers the upper and mid-torso areas. The fifth back compression zone (4012b) is between the right back side (40081b) of the fourth compression zone and the left back side (4008rb) of the fourth compression zone. The fifth back compression zone (4012b) is directly or indirectly joined with the right back side (40081b) of the fourth compression zone and the left back side (4008rb) of the fourth compression zone by a seamless transition. In an alternative embodiment, the back side of the garment can include at least one back compression zone located between the back right side and the back left side of the garment, and the at least one back compression zone can have an amount of compression greater than the first compression zone, or a fabric weight greater than the first fabric weight. In another alternative embodiment, the entire back side of the garment includes a single compression zone, and the single compression zone can have an amount of compression greater than the first compression zone, or a fabric weight greater than the first fabric weight.

In an aspect, one or more of the first compression zone, the second compression zone, the third compression zone, the fourth compression zone, the fifth compression zone, and the sixth compression zone of the single engineered warp knitted component (4001) include the jacquard knit stitch construction. In an aspect, two or more of the first compression zone, the second compression zone, the third compression zone, the fourth compression zone, the fifth compression zone, the sixth compression zone, and the back top compression zone include the jacquard knit stitch construction. In an aspect, three or more of the first compression zone, the second compression zone, the third compression zone, the fourth compression zone, the fifth compression zone, the sixth compression zone, and the back top compression zone include the jacquard knit stitch construction. In an aspect, four or more of the first compression zone, the second compression zone, the third compression zone, the fourth compression zone, the fifth compression zone, the sixth compression zone, and the back top compression zone includes the jacquard knit stitch construction. In an aspect, five or more of the first compression zone, the second compression zone, the third compression zone, the fourth compression zone, the fifth compression zone, the sixth compression zone, and the back top compression zone include the jacquard knit stitch construction. Each of the first compression zone, the second compression zone, the third compression zone, the fourth compression zone, the fifth compression zone, the sixth compression zone, the back top compression zone each include the jacquard knit stitch construction.

FIGS. 25A and 25B illustrate a full torso garment (5000), which is designed to cover the upper torso, the mid-torso, and the lower torso. The full torso garment (5000) is similar to the half torso garment (4000), but the full torso garment (5000) is longer to cover the mid-torso and lower torso. As a result, the fourth compression zone (a fourth right compression zone (5008r) and a fourth left compression zone (50081)) and the fifth compression zone (5012f and 5012b) of the single engineered warp knitted component (5001) of full torso garment (5000) are longer than the corresponding zones in the single engineered warp knitted component (5001) of the half torso garment (4000). In addition, other zones are present that cover the hip area, pelvic area, perennial area, and upper buttocks area. As illustrated, the bottom portion of the full torso garment (5000) has a thong-cut, although a bikini-cut bottom portion or a shorts-cut bottom portion can be included instead.

The upper portion (e.g., bra portion) of the single engineered warp knitted component (5001) of full torso garment (5000) is similar to or the same as the single engineered warp knitted component of garment (3000). In this regard, the description and discussion regarding the single engineered warp knitted component (3001), including first right compression zone (3002r), the first left compression zone (3002l), the second right compression zone (3004r), the second left compression zone (3004l), the second middle compression zone (3004m), the third compression zone (3006), the sixth right compression zone (3014r), the sixth left compression zone (30141), the right side strap (3024r), the left side strap (30241), and the back top compression zone (3016), all apply to the full torso garment (5000) and to the single engineered warp knitted component (5001) of full torso garment (5000) and are not repeated in the discussion regarding the full torso garment (5000) and its single engineered warp knitted component (5001). The following discussion regarding the single engineered warp knitted component (5001) of full torso garment (5000) and full torso garment (5000) are directed to additional features of the single engineered warp knitted component (5001) of full torso garment (5000) and full torso garment (5000).

The full torso garment (5000) includes a single engineered warp knitted component (5001) having a front side (FIG. 25A) and a rear side (FIG. 25B). The front side is sized and shaped to cover an area corresponding to a front portion of a wearer's upper, mid-, and lower torso and the rear side is sized and shaped to cover an area corresponding to a rear portion of a wearer's upper, mid-, and lower torso, but as illustrated includes a thong so that the buttocks are not covered by the garment (5000). The front and rear sides comprise a single engineered warp knitted component (5001) having a plurality of integrated compression zones, where multiple compression zones have different amounts of compression and fabric weight. In an aspect, two or more compression zones can have the same amounts of compression and fabric weight.

In the embodiment illustrated in FIGS. 25A and 25B, the single engineered warp knitted component (5001) of garment (5000) can also include a fourth compression zone having a fourth amount of compression and a fourth fabric weight. The fourth compression zone includes a fourth right compression zone (5008r) and a fourth left compression zone (50081). The fourth right compression zone (5008r) is sized and shaped to cover at least a portion of an area corresponding to a wearer's front, mid-torso, and lower torso below the right breast area and side area. The fourth right compression zone (5008r) is below both the first right compression zone (3002r) and the second right compression zone (3004r). The fourth right compression zone (5008r) extends in width at least along the bottom of the second right compression zone (3004r) and extends from a right front side to a right back side (50081b). The fourth left compression zone (50081) is sized and shaped to cover at least a portion of an area corresponding to a wearer's front, mid-torso, and lower torso below the left breast area and side area. The fourth left compression zone (50081) is below both the first left compression zone (3002l) and the second left compression zone (3004l). The fourth left compression zone (50081) extends in width at least along the bottom of the second left compression zone (3004l) and extends from a left front side to a left back side (5008rb). In another embodiment, the fourth compression zone can be a single zone including a fourth right compression zone, a fourth left compression zone, and a fourth middle compression zone between the fourth right compression zone and the fourth left compression zone. In another embodiment, the garment or single engineered warp knitted component (5001) of the garment includes one or more fourth compression zones in which the one or more fourth compression zones are positioned differently than as illustrated in FIG. 25A or 25B. In yet another embodiment, the garment or single engineered warp knitted component (5001) does not include a fourth compression zone having a different level of compression, a different fabric weight, a different jacquard knit stitch construction, or any combination thereof, as compared the first, second and third compression zones.

In regard to how the various zones of the single engineered warp knitted component (5001) are connected in FIGS. 25A and 25B, the following provides embodiments on how the various zones can be connected. The fourth right compression zone (5008r) is directly or indirectly joined with the second right compression zone (3004r) by a seamless transition and is directly or indirectly joined with the third compression zone (3006) by a seamless transition. The fourth left compression zone (50081) is directly or indirectly joined with the distal of the second left compression zone (3004l) by a seamless transition and is directly or indirectly joined with the third compression zone (3006) by a seamless transition. The fourth right and left compression zones (5008r and 50081) can comprise jacquard knit stitch construction and can include an elastane jacquard knit stitch construction. The elastane jacquard knit stitch construction is configured to engineer a density or a fabric weight in the fourth right compression zone (5008r) and fourth left compression zone (50081). The jacquard knit stitch construction and/or the elastane jacquard knit stitched construction can be independently knitted and can be seamlessly added to the first (ground) knit construction using the second jacquard bar (920).

In regard to the amount of compression of the fourth compression zone, as illustrated in FIG. 25A, the fourth right compression zone (5008r) and the fourth left compression zone (50081) both have the fourth amount of compression. In embodiments where the side compression zones extend from the front of the garment to the back of the garment as illustrated in FIG. 25B, the fourth right compression zone (5008r) extends into the fourth right back compression zone (50081b), the fourth left compression zone (50081) extends into the fourth left back compression zone (5008rb), and each have the fourth amount of compression. The fourth amount compression is greater than the first amount of compression, the fourth amount of compression is greater than the second amount of compression, and the fourth amount of compression is greater than the third amount of compression. Alternative embodiments may have different relationships between the amounts of compression of the various compression zones.

In regard to the fabric weight for the first and fourth zones, as illustrated in FIG. 25A, the fourth right compression zone (5008r) and the fourth left compression zone (50081) both have the fourth fabric weight, and the fourth fabric weight is greater than the first fabric weight, the fourth fabric weight is greater than the second fabric weight, and the fourth fabric weight is greater than the third fabric weight. In one aspect, a difference between the fourth fabric weight and the first fabric weight ranges from 20 GSM to 220 GSM. Alternative embodiments may have different relationships between the fabric weights of the various compression zones.

In the embodiment illustrated in FIGS. 25A and 25B, the single engineered warp knitted component (5001) of garment (5000) also includes a fifth compression zone (5012f) on the front side having a fifth amount of compression and a fifth fabric weight. The fifth compression zone (5012f) covers the upper, mid-, and lower torso. The fifth compression zone (5012f) is below at least a portion of the first right compression zone (3002r), at least a portion of the first left compression zone (3002l), at least a portion of the second right compression zone (3004r), at least a portion of the second left compression zone (3004l), and at least a portion of the third compression zone (3006) and extends laterally at least between the fourth right compression zone (5008r) and the fourth left compression zone (50081). In another embodiment, the fourth compression zone can include the fourth right compression zone, the fourth left compression zone, and the area occupied by the fifth compression zone on the front side as described above, in which the entire fourth compression zone has the fourth amount of compression and the fourth fabric weight. Optionally, the fourth right compression zone (5008r) and the fourth left compression zone (50081) can each individually extend from a front side of the single engineered warp knitted component (5001) of the garment to a back side of the garment. In another embodiment, the single engineered warp knitted component (5001) of the garment includes only a fifth front compression zone present on a side of the single engineered warp knitted component (5001) configured to form a front side of the garment, or includes the fifth back compression zone present only on a side of the engineered warp knitted component (5001) configured to form a back side of the garment. In another embodiment, the garment or single engineered warp knitted component (5001) of the garment includes a fifth compression zone in which the fifth compression zone is positioned differently than as illustrated in FIG. 25A or 25B. In yet another embodiment, the garment or single engineered warp knitted component (5001) does not include a fifth compression zone having a different level of compression, a different fabric weight, a different jacquard knit stitch construction, or any combination thereof, as compared to the first, second, third and fourth compression zones.

In the embodiment illustrated in FIGS. 25A and 25B, the fifth compression zone (5012f) is directly or indirectly joined with the third compression zone (3006) by a seamless transition. The fifth compression zone (5012f) can be directly or indirectly joined with the fourth left compression zone (50081) by a seamless transition and the fourth right compression zone (5008r) by a seamless transition.

In regard to the amount of compression of the fifth compression zone, as illustrated in FIGS. 25A and 25B, the fifth amount of compression of the fifth compression zone is greater than the first amount of compression, the fifth amount of compression is greater than the second amount of compression, the fifth amount of compression is greater than the third amount of compression, and the fifth amount of compression is greater than the fourth amount of compression. Alternative embodiments may have different relationships between the amounts of compression of the various compression zones.

In regard to the fabric weight for the first and fifth compression zones (3002r/l and 5012f) as illustrated in FIG. 25A, the first fabric weight is less than the fifth fabric weight. In an aspect, a difference between the fifth fabric weight and the first fabric weight can range from 20 GSM to 220 GSM. Alternative embodiments may have different relationships between the fabric weights of the various compression zones.

In the embodiment illustrated in FIGS. 25A and 25B, the single engineered warp knitted component (5001) of garment (5000) also has a fifth back compression zone (5012b) on the back side having the fifth amount of compression and the fifth fabric weight. The fifth back compression zone (5012b) covers the upper, mid-, and lower torso areas. The fifth back compression zone (5012b) is between (medial edges) the right back side (50081b) of the fourth compression zone and the left back side (5008rb) of the fourth compression zone. The fifth back compression zone (5012b) is directly or indirectly joined with the right back side (50081b) of the fourth compression zone and the left back side (5008rb) of the fourth compression zone by a seamless transition. In an alternative embodiment, the back side of the garment can include at least one back compression zone located between the back right side and the back left side of the garment, and the at least one back compression zone can have an amount of compression greater than the first compression zone, or a fabric weight greater than the first fabric weight. In another alternative embodiment, the entire back side of the garment includes a single compression zone, and the single compression zone can have an amount of compression greater than the first compression zone, or a fabric weight greater than the first fabric weight.

In the embodiment illustrated in FIGS. 25A and 25B, the single engineered warp knitted component (5001) of garment (5000) includes a hip compression zone (right hip compression zone 5032r and left hip compression zone 50321). As illustrated, the hip compression zone has the second amount of compression and the second fabric weight. Alternatively, the amount of compression of the hip compression zone can be greater than the first amount of compression, and the fabric weight of the hip compression zone can be greater than the first fabric weight. The hip compression zone can have the third amount of compression and the third fabric weight, or can have the fourth amount of compression and the fourth fabric weight, or can have the fifth amount of compression and the fifth fabric weight, or can have the sixth amount of compression and the sixth fabric weight. The hip compression zone includes the right hip compression zone 5032r and the left hip compression zone 50321. The right hip compression zone 5032r is on the bottom side of the fourth compression zone 5008r and side of the fifth compression zone 5012f. The right hip compression zone 5032r is directly or indirectly joined to the bottom of the fourth compression zone 5008r by a seamless transition and directly or indirectly joined to the side of the fifth compression zone 5012f by a seamless transition. The right hip compression zone 5032r and the left hip compression zone 50321 can each have a jacquard knit stitch construction.

In the embodiment illustrated in FIGS. 25A and 25B, the single engineered warp knitted component (5001) of garment (5000) includes pelvic compression zone (5034) having a third amount of compression and a third fabric weight. The pelvic compression zone (5034) is on the bottom of the fifth compression zone (5012f) and the side of the right hip compression zone (5032r) and the side of the left hip compression zone (50321). The pelvic compression zone (5034) is directly or indirectly joined to the distal edge of the fifth compression zone (5012f) by a seamless transition and directly or indirectly joined with the right hip compression zone (5032r) and the left hip compression zone (50321), each by a seamless transition. The pelvic compression zone (5034) can have a jacquard knit stitch construction.

In the embodiment illustrated in FIGS. 25A and 25B, the single engineered warp knitted component (5001) of garment (5000) includes a thong (5036) having a first amount of compression and a first fabric weight. The thong (5036) is on the bottom of the fifth back compression zone (5012b) and the bottom of the pelvic compression zone (5034). The thong (5036) is directly or indirectly joined with the fifth back compression zone (5012b) by a seamless transition and is directly or indirectly joined with the pelvic compression zone (5034) by a seamless transition. The thong (5036) can have a jacquard knit stitch construction.

In an aspect, one or more of the first compression zone, the second compression zone, the third compression zone, the fourth compression zone, the fifth compression zone, the sixth compression zone, hip compression zone, and pelvic compression zone of the single engineered warp knitted component (5001) include the jacquard knit stitch construction. In an aspect, two or more of the first compression zone, the second compression zone, the third compression zone, the fourth compression zone, the fifth compression zone, the sixth compression zone, hip compression zone, and pelvic compression zone include the jacquard knit stitch construction. In an aspect, three or more of the first compression zone, the second compression zone, the third compression zone, the fourth compression zone, the fifth compression zone, the sixth compression zone, hip compression zone, and pelvic compression zone include the jacquard knit stitch construction. In an aspect, four or more of the first compression zone, the second compression zone, the third compression zone, the fourth compression zone, the fifth compression zone, the sixth compression zone, hip compression zone, and pelvic compression zone include the jacquard knit stitch construction. In an aspect, five or more of the first compression zone, the second compression zone, the third compression zone, the fourth compression zone, the fifth compression zone, the sixth compression zone, hip compression zone, and pelvic compression zone include the jacquard knit stitch construction. In an aspect, six or more of the first compression zone, the second compression zone, the third compression zone, the fourth compression zone, the fifth compression zone, the sixth compression zone, hip compression zone, and pelvic compression zone include the jacquard knit stitch construction. Each of the first compression zone, the second compression zone, the third compression zone, the fourth compression zone, the fifth compression zone, the sixth compression zone, hip compression zone, and pelvic compression zone each include the jacquard knit stitch construction.

FIGS. 26A and 26B illustrate a full torso garment (6000) including a single engineered warp knitted component (6001) with shorts, which is designed to cover the upper torso, the mid-torso, the lower torso, and the upper to mid-thigh. The single engineered warp knitted component (6001) of full torso garment (6000) and full torso garment (6000) are similar to the single engineered warp knitted component (3001) of full torso garment (5000) and full torso garment (5000) shown in FIGS. 25A and 25B, but the single engineered warp knitted component (6001) of full torso garment (6000) and full torso garment (6000) cover the upper to mid-thigh and the entire buttocks area.

The upper portion (e.g., bra portion) of the single engineered warp knitted component (6001) of full torso garment (6000) and full torso garment (6000) are similar to or the same as the single engineered warp knitted component (6001) of garment (3000) and garment (3000). In this regard, the description and discussion regarding the first right compression zone (3002r), the first left compression zone (3002l), the second right compression zone (3004r), the second left compression zone (3004l), the second middle compression zone (3004m), the third compression zone (3006), the sixth right compression zone (3014r), the sixth left compression zone (30141), the right side strap (3024r), the left side strap (30241), and the back top compression zone (3016) apply to the single engineered warp knitted component (6001) of full torso garment (6000) and to full torso garment (6000) and are not repeated in the discussion regarding the single engineered warp knitted component (6001) of full torso garment (6000) and full torso garment (6000). The following discussion regarding the single engineered warp knitted component (6001) of full torso garment (6000) and full torso garment (6000) are directed to addition features of the single engineered warp knitted component (6001) of full torso garment (6000) and the full torso garment (6000).

The single engineered warp knitted component (6001) of full torso garment (6000) includes a front side (FIG. 26A) and a rear side (FIG. 26B). The front side is sized and shaped to cover an area corresponding to a front portion of a wearer's upper, mid-, and lower torso and the rear side is sized and shaped to cover an area corresponding to a rear portion of a wearer's upper, mid-, and lower torso, but as illustrated also includes shorts that cover the upper to mid-thigh. The front and rear sides comprise a single engineered warp knitted component (6001) having a plurality of integrated compression zones, where multiple compression zones have different amounts of compression and fabric weight. In an aspect, two or more compression zones can have the same amounts of compression and fabric weight.

In the embodiment illustrated in FIGS. 26A and 26B, the single engineered warp knitted component (6001) of garment (6000) can also include a fourth compression zone having a fourth amount of compression and a fourth fabric weight. The fourth compression zone includes a fourth right compression zone (6008r) and a fourth left compression zone (6008l). The fourth right compression zone (6008r) is sized and shaped to cover at least a portion of an area corresponding to a wearer's front, mid-, and lower torso below the right breast area as well on hip and the side and back of the right upper to mid-thighs. The fourth right compression zone (6008r) is below both the first right compression zone (3002r) and the second right compression zone (3004r). The fourth right compression zone (6008r) extends in width along the bottom of the second right compression zone (3004r) and extends from a right front side to a right back side (6008lb). The fourth left compression zone (6008l) is sized and shaped to cover at least a portion of an area corresponding to a wearer's front, mid-, lower torso below the left breast area as well the hip and on the side and back of the right upper to mid-thighs. The fourth left compression zone (6008l) is below both the first left compression zone (3002l) and the second left compression zone (3004l). The fourth left compression zone (6008l) extends in width along the bottom of the second left compression zone (3004l) and extends from a left front side to a left back side (6008rb). In another embodiment, the fourth compression zone can be a single zone including a fourth right compression zone (6008r), a fourth left compression zone (6008l), and a fourth middle compression zone between the fourth right compression zone (6008r) and the fourth left compression zone (6008l). In another embodiment, the garment or single engineered warp knitted component (6001) of the garment includes one or more fourth compression zones in which the one or more fourth compression zones are positioned differently than as illustrated in FIG. 26A or 26B. In yet another embodiment, the garment or single engineered warp knitted component (6001) does not include a fourth compression zone having a different level of compression, a different fabric weight, a different jacquard knit stitch construction, or any combination thereof, as compared to the first, second and third compression zones.

In regard to how the various zones of the single engineered warp knitted component (6001) are connected in FIGS. 26A and 26B, the following provides embodiments on how the various zones can be connected. The fourth right compression zone (6008r) is directly or indirectly joined to the bottom of the second right compression zone (3004r) by a seamless transition and is directly or indirectly joined with the third compression zone (3006) by a seamless transition. The fourth left compression zone (6008l) is directly or indirectly joined to the bottom of the second left compression zone (3004l) by a seamless transition and is directly or indirectly joined with the third compression zone (3006) by a seamless transition. The fourth right compression zone (6008r) and fourth left compression zone (6008l) can comprise jacquard knit stitch construction and can include an elastane jacquard knit stitch construction. The elastane jacquard knit stitch construction is configured to engineer a density or a fabric weight in the fourth right compression zone (6008r) and fourth left compression zone (6008l). The jacquard knit stitch construction and/or the elastane jacquard knit stitched construction can be independently knitted and can be seamlessly added to the first (ground) knit construction using the second jacquard bar 920.

In regard to the amount of compression of the fourth compression zone, as illustrated in FIG. 26A, the fourth right compression zone (6008r) and the fourth left compression zone (6008l) both have the fourth amount of compression. In embodiments where the side compression zones extend from the front of the garment to the back of the garment as illustrated in FIG. 26B, the fourth right compression zone (6008r) extends into the fourth right back compression zone (6008lb), the fourth left compression zone (6008l) extends into the fourth left back compression zone (6008rb), and each have the fourth amount of compression. The fourth amount compression is greater than the first amount of compression, the fourth amount of compression is greater than the second amount of compression, and the fourth amount of compression is greater than the third amount of compression. Alternative embodiments may have different relationships between the amounts of compression of the various compression zones.

In regard to the fabric weight for the first and fourth zones, as illustrated in FIG. 26A, the fourth right compression zone (6008r) and the fourth left compression zone (6008l) both have the fourth fabric weight, and the fourth fabric weight is greater than the first fabric weight, the fourth fabric weight is greater than the second fabric weight, and the fourth fabric weight is greater than the third fabric weight. In one aspect, a difference between the fourth fabric weight and the first fabric weight ranges from 20 GSM to 220 GSM. Alternative embodiments may have different relationships between the fabric weights of the various compression zones.

In the embodiment illustrated in FIGS. 26A and 26B, the single engineered warp knitted component (6001) of garment (6000) also includes a fifth compression zone (6012f) on the front side having a fifth amount of compression and a fifth fabric weight. The fifth compression zone (6012f) covers the upper, mid-, and lower torso. The fifth compression zone (6012f) is below at least a portion of the first right compression zone (3002r), at least a portion of the first left compression zone (3002l), at least a portion of the second right compression zone (3004r), at least a portion of the second left compression zone (3004l), and at least a portion of the third compression zone (3006) and extends laterally at least between the fourth right compression zone (6008r) and the fourth left compression zone (6008l). In another embodiment, the fourth compression zone can include the fourth right compression zone (6008r), the fourth left compression zone (6008l), and the area occupied by the fifth compression zone (6012f) on the front side as described above, in which the entire fourth compression zone has the fourth amount of compression and the fourth fabric weight. Optionally, the fourth right compression zone (6008r) and the fourth left compression zone (6008l) can each individually extend from a front side of the garment to a back side of the garment. In yet another embodiment, the garment does not include a fifth compression zone.

In an aspect, the fifth compression zone (6012f) is directly or indirectly joined with the third compression zone (3006) by a seamless transition. The fifth compression zone (6012f) can be directly or indirectly joined with the fourth left compression zone (6008l) by a seamless transition and the fourth right compression zone (6008r) by a seamless transition.

In regard to the amount of compression of the fifth compression zone, as illustrated in FIG. 26A, the fifth amount of compression of the fifth compression zone (6012f) is greater than the first amount of compression, the fifth amount of compression is greater than the second amount of compression, the fifth amount of compression is greater than the third amount of compression, and the fifth amount of compression is greater than the fourth amount of compression. Alternative embodiments may have different relationships between the amounts of compression of the various compression zones.

In regard to the fabric weight for the first and fifth compression zones (3002r/l and 6012f) as illustrated in FIG. 26A, the first fabric weight is less than the fifth fabric weight. In an aspect, a difference between the fifth fabric weight and the first fabric weight can range from 20 GSM to 220 GSM. Alternative embodiments may have different relationships between the fabric weights of the various compression zones.

In the embodiment illustrated in FIGS. 26A and 26B, the single engineered warp knitted component (6001) of garment (6000) also has a fifth back compression zone (6012b) on the back side having the fifth amount of compression and the fifth fabric weight. The fifth back compression zone (6012b) covers the upper, mid-, and lower torso areas. The fifth back compression zone (6012b) is between the right back side (6008lb) of the fourth compression zone and the left back side (6008rb) of the fourth compression zone. The fifth back compression zone (6012b) is directly or indirectly joined with the right back side (6008lb) of the fourth compression zone and the left back side (6008rb) of the fourth compression zone by a seamless transition. In an alternative embodiment, the back side of the garment can include at least one back compression zone located between the back right side and the back left side of the garment, and the at least one back compression zone can have an amount of compression greater than the first compression zone, or a fabric weight greater than the first fabric weight. In another alternative embodiment, the entire back side of the garment includes a single compression zone, and the single compression zone can have an amount of compression greater than the first compression zone, or a fabric weight greater than the first fabric weight.

In the embodiment illustrated in FIGS. 26A and 26B, the single engineered warp knitted component (6001) of garment (6000) includes pelvic compression zone (6034) having a third amount of compression and a third fabric weight. As shown in FIG. 26A, the pelvic compression zone (6034) has a “M” shape and is sized and shaped to cover the pelvic region, extends up to the front hip area (left and right) and then extends down along the front of each thigh of a wearer. Other configurations of pelvic compression zone are contemplated, including configurations in which the pelvic compression zone is configured to extend across a pelvic area and to right and left hip areas of a wearer. Alternatively, the single engineered warp knitted component (6001) may not include a pelvic compression zone. The pelvic compression zone (6034) is on the bottom of the fifth compression zone (6012f) and the side of the right fourth compression zone (6008r) and the side of the left fourth compression zone (6008l). The pelvic compression zone (6034) is directly or indirectly joined to the bottom of the fifth compression zone (6012f) by a seamless transition and directly or indirectly joined to the side of the right fourth compression zone (6008r) and the side of the left fourth compression zone (6008l), each by a seamless transition. The pelvic compression zone (6034) can have a jacquard knit stitch construction.

In regard to the amount of compression of the pelvic compression zone (6034), as illustrated in FIG. 26A, the amount of compression of the pelvic compression zone (6034) is greater than the first amount of compression, and is the same as the third amount of compression. Alternative embodiments may have different relationships between the amounts of compression of the various compression zones.

In regard to the fabric weight for the pelvic compression zone (6034) as illustrated in FIG. 26A, the fabric weight of the pelvic compression zone is greater than the first fabric weight, and is the same as the third fabric weight. In an aspect, a difference between the third fabric weight and the first fabric weight can range from 20 GSM to 220 GSM. Alternative embodiments may have different relationships between the fabric weights of the various compression zones.

In the embodiment illustrated in FIGS. 26A and 26B, the single engineered warp knitted component (6001) of garment (6000) includes a thigh compression zone sized and shaped to cover a thigh area of a wearer. The thigh area includes a right thigh area (6042r) and a left thigh area (60421). In an aspect, the thigh area can include the upper thigh and/or the mid-thigh area. In another aspect, the thigh area can extend to just above (e.g., within about 2 inches, about 1 inch or about 0.5 inch) the knee. The thigh compression zone has a seventh amount of compression and a seventh fabric weight. The right thigh area (6042r) is sized and shaped to cover the front of the right thigh area of a wearer. As illustrated in FIG. 26A, the right thigh area (6042r) is within an area that is substantially surrounded by the pelvic compression zone (6034). Embodiments in which the thigh compression zone is positioned below the pelvic compression zone are also contemplated. The right thigh area (6042r) is directly or indirectly joined to of the right side of the pelvic compression zone (6034) by a seamless transition. The left thigh area (60421) is sized and shaped to cover the front of the left upper thigh area of the wearer. The left thigh area (60421) is within an area that is substantially surrounded by the pelvic compression zone (6034). The left thigh area (60421) is directly or indirectly joined to the left side of the pelvic compression zone (6034) by a seamless transition.

In regard to the amount of compression of the thigh compression zone (6042r/l), as illustrated in FIG. 26A, the amount of compression of the thigh compression zone (6042r/l) is greater than the first amount of compression, and is less than the fifth amount of compression. Alternative embodiments may have different relationships between the amounts of compression of the various compression zones.

In regard to the fabric weight for the thigh compression zone (6042r/l) as illustrated in FIG. 26A, the fabric weight of the thigh compression zone is greater than the first fabric weight, and is less than the fifth fabric weight. In an aspect, a difference between the fifth fabric weight and the first fabric weight can range from 20 GSM to 220 GSM. Alternative embodiments may have different relationships between the fabric weights of the various compression zones.

In the embodiment illustrated in FIGS. 26A and 26B, the single engineered warp knitted component (6001) of garment (6000) includes a seventh compression zone that has a first amount of compression and a first fabric weight. The seventh compression zone is configured to cover a buttocks area of a wearer and includes a right buttocks area (6032r) and a left buttocks area (600321), where each buttocks area is circular or oval in shape and is surrounded by the by the right and left back side of the fourth compression zones (6008lb and 6008rb). The right buttocks area (6032r) is directly or indirectly joined with the right and left back side of the fourth compression zones (6008lb and 6008rb) by a seamless transition. The left buttocks area (60321) is directly or indirectly joined with the right and left back side of the fourth compression zones (6008lb and 6008rb) by a seamless transition.

In regard to the amount of compression of the seventh compression zone (6032r/l), as illustrated in FIG. 26B, the amount of compression of the seventh compression zone (6032r/l) is the same as the first amount of compression, and is less than the fifth amount of compression. Alternative embodiments may have different relationships between the amounts of compression of the various compression zones.

In regard to the fabric weight for the seventh compression zone (6032r/l) as illustrated in FIG. 26B, the fabric weight of the seventh compression zone is the same as the first fabric weight, and is less than the fifth fabric weight. In an aspect, a difference between the fifth fabric weight and the first fabric weight can range from 20 GSM to 220 GSM. Alternative embodiments may have different relationships between the fabric weights of the various compression zones.

In the embodiment illustrated in FIGS. 26A and 26B, the single engineered warp knitted component (6001) of garment (6000) can optionally include an eighth compression zone that has a first amount of compression and a first fabric weight. The eighth compression zone includes a right leg area (6046r) that is along the bottom of the right fourth compression zone (6008r). The right leg area (6046r) is directly or indirectly joined with the right fourth compression zones (6008r) by a seamless transition. The eighth compression zone includes a left leg area (6046l) that is along the bottom of the left fourth compression zone (6008l). The left leg area (6046l) is directly or indirectly joined with the left fourth compression zones (6008l) by a seamless transition. In an alternative embodiment, the eighth compression zone runs along the bottom of a right or left leg portion of the garment, is directly or indirectly joined with a leg compression zone sized and shaped to cover a left or right leg area of a wearer, and directly or indirectly connects to the second compression zone of the engineered warp knitted component (6001) of the garment (6000).

In an aspect, one or more of the first compression zone, the second compression zone, the third compression zone, the fourth compression zone, the fifth compression zone, the sixth compression zone, the seventh compression zone, and the eighth compression zone include the jacquard knit stitch construction. In an aspect, two or more of the first compression zone, the second compression zone, the third compression zone, the fourth compression zone, the fifth compression zone, the sixth compression zone, the seventh compression zone, and the eighth compression zone include the jacquard knit stitch construction. In an aspect, three or more of the first compression zone, the second compression zone, the third compression zone, the fourth compression zone, the fifth compression zone, the sixth compression zone, the seventh compression zone, and the eighth compression zone include the jacquard knit stitch construction. In an aspect, four or more of the first compression zone, the second compression zone, the third compression zone, the fourth compression zone, the fifth compression zone, the sixth compression zone, the seventh compression zone, and the eighth compression zone include the jacquard knit stitch construction. In an aspect, five or more of the first compression zone, the second compression zone, the third compression zone, the fourth compression zone, the fifth compression zone, the sixth compression zone, the seventh compression zone, and the eighth compression zone include the jacquard knit stitch construction. In an aspect, six or more of the first compression zone, the second compression zone, the third compression zone, the fourth compression zone, the fifth compression zone, the sixth compression zone, the seventh compression zone, and the eighth compression zone include the jacquard knit stitch construction. Each of the first compression zone, the second compression zone, the third compression zone, the fourth compression zone, the fifth compression zone, the sixth compression zone, the seventh compression zone, and the eighth compression zone each include the jacquard knit stitch construction.

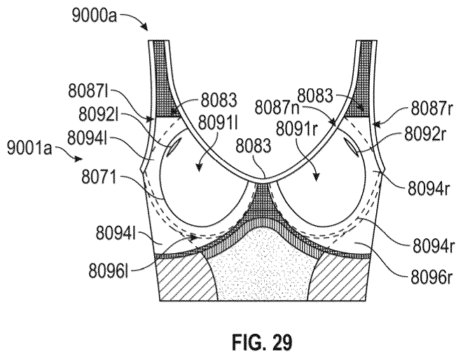

Each of the garments described in FIGS. 23A-26B can optionally include a liner (which is not part of the single engineered warp knitted component) sized and shaped to cover all or a part of the right and left breast areas of the garment, such as all of or a portion of the first right compression zone and the first left compression zone of the single engineered warp knitted component (3001, 4001, 5001, 6001). FIGS. 27A, 28A, and 29 illustrate an upper torso garment (8000a, 8000b, 9000). FIG. 27A illustrates the inner surface of the front of the upper torso garment (8000a), FIG. 28A illustrates the inner surface of upper torso garment (8000b). FIG. 29 illustrates the inner surface of upper torso garment (9000). In an aspect shown in FIGS. 27A and 28A, the liner (8096) can be sized and shaped to cover at least the right and left breast areas, while in FIG. 29 liners (8096l and 8096r) are sized and shaped to cover the left breast area and the right breast area, respectively. As shown in FIGS. 27A and 28A, the liner (8096) is a single component sized and shaped to cover at least a portion of both the right and left breast areas. As shown in FIG. 29, the liners (8096r and 8096l) comprise a right liner (8096r) sized and shaped to cover at least a portion of the right breast area, and a left liner (8096l) sized and shaped to cover at least a portion of the left breast area. The right liner (8096r) can be sized and shaped to cover at least a portion of a first right compression area of the single engineered warp knitted component (9001), and the left liner (8096l) can be sized and shaped to cover at least a portion of a first left compression area.

As illustrated in FIGS. 27A and 28A, the liner (8096) is on the inside of the garment, meaning the liner (8096) forms the innermost layer of the garment and is configured to be positioned directly opposite the body of a wearer. Similarly, FIG. 29 illustrates the right liner (8096r) and the left liner (8096l) are on the inside of the garment, meaning the right liner (8096r) and the left liner (8096l) each forms the innermost layer of the garment and is configured to be positioned directly opposite the body of a wearer. In another aspect that is not illustrated, the liner (8096) can be on the outside of the garment, meaning the liner forms the outermost layer of the garment and is configured to be positioned facing away from the body of a wearer. The liner (8096) can comprise a naturally-occurring material, a regenerated natural material, a synthetic material, or any combination thereof. The liner (8096) can be made of a material such as cotton, silk, modal, micro-modal, rayon, lyocell, viscose, cupro, artificial silk, nylon, polypropylene or any other natural or a synthetic material or any combination thereof. In some embodiments the liner (8096) can comprise an elastic material including elastane yarn. The liner (8096) can be a knit fabric, a woven fabric, or a non-woven fabric. In one aspect, the liner (8096) is a knit nylon fabric.

The liner (8096, 8096r and 8096l) can be affixed (e.g., thermally bonded or adhesively bonded or stitched) to the single engineered warp knitted component (8001a, 8001b, 9001) of the upper torso garment (8000a, 8000b, 9000), creating affixed areas (8094, 80941, 8094r) in which the liner (8096, 8096r and 8096l) and the single engineered warp knitted component (8001a, 8001b, 9000) are affixed to each other. In an aspect, the liner (8096, 8096r and 8096l) can be affixed to the single engineered warp knitted component (8001a, 8001b, 9001) at least around a portion of an outer perimeter of the liner (8096, 8096r and 8096l). In one aspect, the single engineered warp knitted component (8001a, 8001b, 9000) and the liner (8096, 8096r and 8096l) can be affixed to each other in affixed areas (8094, 80941, 8094r) which extend over a surface area located within the outer perimeter of the lining, forming an affixed surface area affixed (8094, 80941, 8094r) extending inside the outer perimeter of the liner (8096, 8096r and 8096l) over which opposing surfaces of the liner (8096, 8096r and 8096l) and the single engineered warp knitted component (8001a, 8001b, 9001) are affixed to each other. In an unillustrated aspect, the affixed area (8094, 80941, 8094r) can comprise substantially the entire surface area of the liner (8096, 8096r and 8096l). In another aspect as illustrated in FIGS. 27A-29, an inner area (8091, 8091l, and 8091r) of the liner (8096, 8096r and 8096l) positioned inside the outer perimeter can be an unaffixed area (e.g., unbonded area) (8091, 8091l, and 8091r), meaning that in the unbonded area (8091, 8091l, and 8091r) the surfaces of the liner (8096, 8096r and 8096l) and the single engineered warp knitted component (8001a, 8001b, 9001) directly opposite each other are not bonded to each other. In other words, in this aspect, along the surface area of the single engineered warp knitted component (8001a, 8001b, 9001) covered by the liner (8096, 8096r and 8096l), the liner is affixed to the single engineered warp knitted component (8001a, 8001b, 9001) in an affixed area (8094, 80941, 8094r) at least along the outer perimeter of the liner, and the affixed area (8094, 80941, 8094r) extends inward from the outer perimeter up to a perimeter of an unbonded area (8091, 8091l, and 8091r), forming an unaffixed (e.g., unbonded) space or pocket between the inner surfaces of liner and the opposing surface of the single engineered knitted component in the unaffixed area (8091, 8091l, and 8091r). Affixing the liner (8096, 8096r and 8096l) around at least a portion of its outer perimeter creates this unaffixed area (8091, 8091l, and 8091r), such as the unaffixed space or pocket. In the embodiment shown in FIGS. 27A-29, the affixed area (8094, 80941, 8094r) extends from the outer perimeter of the liner (8096, 8096r and 8096l) to a perimeter of the unaffixed area (8071).

As illustrated in FIGS. 27A and 28A, the liner (8096) is affixed to at least the top and bottom of the of the first right and first left compression zones of the single engineered warp knitted component (8001a, 8001b) of the garment in an affixed area (8094) which is outside the perimeter of the unaffixed area (8071), leaving an unaffixed area (8091) inside the perimeter of the unaffixed area (8071) (note that the seamless transitions between compression zones are shown as dashed lines in FIG. 27A or 28A indicating their positions below the liner (8096) but the compression zones and their seamless transitions are shown in FIGS. 23A, 24A, 25A, and 26A). In the aspect illustrated in FIG. 27A, the garment (8000a) comprises affixed areas (8094) and unaffixed areas (8091) between the liner (8096) and a surface of the single engineered warp knitted component (8001a), and the perimeter of the unaffixed area (8071) is in the first right compression zone and in the first left compression zone. In the aspect illustrated in FIG. 28A, the garment (8000b) comprises affixed areas (8094) and unaffixed areas (8091) between the liner (8096) and a surface of the single engineered warp knitted component (8001b), and the perimeter of the unaffixed area (8071) is along the seamless transition between the first compression zones (i.e., the right compression zone and the first left compression zone) and the compression zone or zones directly adjacent to the first compression zone.

As illustrated in FIG. 29, the right liner and left liner (8096r and 8096l) are each independently affixed to at least the top and bottom of the of the first right and first left compression zones of the single engineered warp knitted component (9001) of the garment (9000) outside the perimeter of the unaffixed area (8071), leaving an unaffixed area (8091r and 8091l) inside the perimeter of the unaffixed area (8071) (note that the compression zones are shown as dashed lines in FIG. 29 but are shown in FIGS. 23A, 24A, 25A, and 26A). In an aspect, the garment (9000) comprises unaffixed areas (8091r and 8091l) between the right liner and left liner (8096r and 8096l), respectively, and a surface of the single engineered warp knitted component (9001) in the first right compression zone and in the first left compression zone, respectively.

Each unaffixed area (8091, 8091r and 8091l) of the garment may include an aperture or slit (8092r and 8092l) configured to allow insertion of a cookie into the volume between the liner (8096, 8096r and 8096l) and the single engineered warp knitted component (8001a, 8001b, 9001) in the unaffixed area unaffixed areas (8091, 8091l and 8091r). As shown in FIG. 28A, a portion of the outer perimeter of the liner (8071, 8071r and 8071l) may remain unaffixed, creating an aperture (8092r and 8092l) through which a cookie can be inserted into the unaffixed area (8091, 8091r and 8091l). Similarly, a perimeter of the unaffixed area (8071, 8071r and 8071l) may include an aperture. Alternatively, either the liner (8096, 8096r, 8096l) or the single engineered warp knitted component (8001a, 8001b, 9001) may include one or more slits within an unaffixed area (8091, 8091r and 8091l) to allow one or more cookies to be inserted into the unaffixed area. As illustrated in FIG. 27A, a right slit (8092r) can be included so a cookie can be inserted into the unaffixed area (8091) between the lining (8096) and the first right compression zone of the single engineered warp knitted component (8001a). As shown in FIG. 27B, the right slit (8092r) can be positioned on any side of the unaffixed area (8091r) and can be sized to allow insertion of a cookie into the unaffixed area (8091). As shown in FIG. 27A, a left slit (8092l) can be included so a cookie can be inserted into the left unaffixed area (8091) between the lining (8096) and the first left compression zone of the single engineered warp knitted component (8001a). As shown in FIG. 27B, the left slit (8092l) can be positioned on any side of the left unbonded area (8091) and can be sized to allow insertion of a cookie into the left unbonded area (8091). While in FIGS. 27A, 28A, and 29 the right slit (8092r) and the left slit (8092l) are positioned in the upper right of the unbonded area (8091) and in the upper left of the unbonded area, respectively, of the upper torso garments (8000a, 8000b, 9000), the right slit and left can be positioned in other areas such as those shown in FIGS. 27B and 28B. Typically the slits are on the inside of the garment, but optionally can be positioned on the outside of the garment. FIGS. 28A and 28B illustrates an embodiment that includes aperture or slit (8092r and 8092l) at the edge of the liner (8096).

In an aspect, the liner (8096) can include an upper edge (8083) and a lower edge (8081). In an aspect, the lower edge (8081) of the liner (8096) can be curved forming a curved edge (8085) between the right breast area and the left breast area, where the curved portion is curving gently upwards towards a wearer's sternum area. In an aspect and as shown in FIG. 28A, the lower edge 8081 is not curved. FIG. 29 illustrates that right liner and left liner (8096r and 8096l) as independent liners one of which (8096r) is sized and shaped to cover the right breast areas area including at least a portion of the first right compression zone, and the other of which (8096l) is sized and shaped to cover the left breast area including at least a portion of the first left compression zone.

In an aspect, along one or more openings in the garment, an outer edge (8087, 8087n, 80871 and 8087r) of the single engineered warp knitted component (8001a, 8001b, 9001) may be folded inwards back on itself and affixed to itself to create a double-layered edge. The double-layered edge (8087, 8087n, 80871 and 8087r) may include a portion of a liner (8096, 8096l, 8096r). As illustrated in FIGS. 27A and 29, the double-layered edge (8087, 8087n) may be along at least a portion of a neck opening of the garment. As illustrated in FIGS. 27A and 29, the double-layered edge (8087) may be along at least a portion of an arm opening of the garment. The double-layered edge (8087) may be along at least a portion of a bottom edge of the garment, such as along at least a portion of a bottom edge of an upper torso garment, of a half torso garment, or of a full torso garment. The double-layered edge (8087) may be along a leg opening of the garment.

While particular elements, embodiments and applications of the present application have been shown and described, it will be understood, that the scope of the application is not limited thereto, since modifications can be made by those skilled in the art without departing from the scope of the present application, particularly in light of the foregoing teachings. Thus, for example, in any method or process disclosed herein, the acts or operations making up the method/process may be performed in any suitable sequence and are not necessarily limited to any particular disclosed sequence. Elements and components can be configured or arranged differently, combined, and/or eliminated in various embodiments. The various features and processes described above may be used independently of one another, or may be combined in various ways. All possible combinations and subcombinations are intended to fall within the scope of this application. Reference throughout this disclosure to “some embodiments,” “an embodiment,” or the like, means that a particular feature, structure, step, process, or characteristic described in connection with the embodiment is included in at least one embodiment. Thus, appearances of the phrases “in some embodiments,” “in an embodiment,” or the like, throughout this disclosure are not necessarily all referring to the same embodiment and may refer to one or more of the same or different embodiments. Indeed, the novel methods and systems described herein may be embodied in a variety of other forms; furthermore, various omissions, additions, substitutions, equivalents, rearrangements, and changes in the form of the embodiments described herein may be made without departing from the spirit of the application.

Various aspects and advantages of the embodiments have been described where appropriate. It is to be understood that not necessarily all such aspects or advantages may be achieved in accordance with any particular embodiment. Thus, for example, it should be recognized that the various embodiments may be carried out in a manner that achieves or optimizes one advantage or group of advantages as taught herein without necessarily achieving other aspects or advantages as may be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without operator input or prompting, whether these features, elements and/or steps are included or are to be performed in any particular embodiment. No single feature or group of features is required for or indispensable to any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The example calculations, simulations, results, graphs, values, and parameters of the embodiments described herein are intended to illustrate and not to limit the disclosed embodiments. Other embodiments can be configured and/or operated differently than the illustrative examples described herein.

What is claimed is:

1. A garment, comprising:
a front side and a rear side, wherein the front side is sized and shaped to cover an area corresponding to a front portion of a wearer's torso, the rear side is sized and shaped to cover an area corresponding to a rear portion of a wearer's torso, and the front and rear sides comprise a single engineered warp knitted component having a plurality of integrated compression zones, the plurality of integrated compression zones including:
a first compression zone on the front side, the first compression zone having a first amount of compression and a first fabric weight,
wherein the first compression zone includes a first right compression zone and a first left compression zone, the first right compression zones includes a region sized and shaped to cover at least a portion of an area corresponding to a wearer's right breast, the first left compression zones includes a region sized and shaped to cover at least a portion of an area corresponding to a wearer's left breast;
a second compression zone on the front side, the second compression zone having a second amount of compression and a second fabric weight,
wherein the second compression zone includes a second right compression zone, a second left compression zone, and a second middle compression zone, the second right compression zone includes a region sized and shaped to cover an area corresponding to a portion of a wearer's torso below the right breast area and the second right compression zone extends in width at least along the bottom of the first right compression zone, the second left compression zone includes a region sized and shaped to cover an area corresponding to a portion of a wearer's torso below the left breast area and the second left compression zone extends at least along the bottom of the first left compression zone, wherein the second middle compression zone includes a region sized and shaped to cover an area between the right breast area and the left breast area and connects the second left compression zone and the second right compression zone;
a third compression zone on the front side, the third compression zone having a third amount of compression and a third fabric weight,
wherein the third compression zone is sized and shaped to cover at least a portion of an area corresponding to a wearer's front torso below both the right and left breast areas and between the right and left breast areas, the third compression zone is below both the first left compression zone and the first right compression zone and extends in width at least between the bottom of the second right compression zone and the bottom of the second left compression zone;
wherein the first right compression zone is directly or indirectly joined to the second right compression zone by a first seamless transition, the first left compression zone is directly or indirectly joined to the second left compression zone by a second seamless transition, the second right compression zone and the second left compression zone are both directly or indirectly joined to the third compression zone by a third seamless transition;
wherein the first amount of compression is less than the second amount of compression and the first amount of compression is less than the third amount of compression;
wherein a difference between the first fabric weight and the second fabric weight or between the first fabric weight and the third fabric weight ranges from 100 grams per square meter (GSM) to 220 GSM, and wherein the first fabric weight is less than the second fabric weight and the first fabric weight is less than the third fabric weight; and
wherein at least one of a) the first right compression zone and the first left compression zone, b) the second right compression zone and the second left compression zone, and c) the third compression zone comprises a jacquard knit stitch construction, wherein the jacquard knit stitch construction includes an elastane jacquard construction, and the elastane jacquard construction is configured to increase a density or configured to increase a fabric weight or configured to increase both a density and a fabric weight.

2. The garment of claim 1, further comprising a right side strap and a left side strap, wherein each of the right side strap and the left side strap extend from the front side to the back side.

3. The garment of claim 1, wherein the plurality of integrated compression zones further comprise:
a fourth compression zone having a fourth amount of compression and a fourth fabric weight,
wherein the fourth compression zone includes a fourth right compression zone and a fourth left compression zone, the fourth right compression zone is sized and shaped to cover at least a portion of an area corresponding to a wearer's front torso below the right breast area, the fourth right compression zone is below both the first right compression zone and the second right compression zone, extends in width at least along the bottom of the second right compression zone, and extends from a right front side to a right back side, and the fourth left compression zone is sized and shaped to cover at least a portion of an area corresponding to a wearer's front torso below the left breast area, the fourth left compression zone is below both the first left compression zone and the second left compression zone, extends in width at least along the bottom of the second left compression zone, and extends from a left front side to a left back side;
wherein the fourth right compression zone is directly or indirectly joined to the lateral edge of the second right compression zone by a seamless transition and is directly or indirectly joined with the third compression zone by a seamless transition, the fourth left compression zone is directly or indirectly joined with the second left compression zone by a seamless transition and is directly or indirectly joined with the third compression zone by a seamless transition; and
wherein a difference between the fourth fabric weight and the first fabric weight ranges from 100 GSM to 220 GSM, wherein the first fabric weight is less than the fourth fabric weight.

4. The garment of claim 1, wherein the second right compression zone, the second left compression zone, the third compression zone, the fourth right compression zone, and the fourth left compression zone each include the jacquard knit stitch construction.

5. The garment of claim 3, wherein the plurality of integrated compression zones further comprise:
a fifth compression zone on the front side having a fifth amount of compression and a fifth fabric weight,
wherein the fifth compression zone is below the first right compression zone, the first left compression zone, the second right compression zone, the second left compression zone, and the third compression zone and extends between the first right compression zone and the first left compression zone;
the fifth compression zone is directly or indirectly joined with the third compression zone by a seamless transition, wherein the fifth compression zone is directly or indirectly joined with the fourth left compression zone by a seamless transition and the fourth right compression zone by a seamless transition; and
wherein a difference between the fifth fabric weight and the first fabric weight ranges from 100 GSM to 220 GSM, wherein the first fabric weight is less than the fifth fabric weight.

6. The garment of claim 5, wherein each of the second right compression zone, the second left compression zone, the third compression zone, the fourth right compression zone, the fourth left compression zone, and the fifth compression zone each include the jacquard knit stitch construction.

7. The garment of claim 5, wherein the plurality of integrated compression zones further comprise:
a fifth back compression zone on the back side having the fifth amount of compression and the fifth fabric weight;
wherein the fifth back compression zone is between the right back side of the fourth compression zone and the left back side of the fourth compression zone;
wherein the fifth back compression zone is directly or indirectly joined with the right back side of the fourth compression zone and the left back side of the fourth compression zone by a seamless transition;
wherein a difference between the fifth fabric weight and the first fabric weight ranges from 100 GSM to 220 GSM, wherein the first fabric weight is less than the fifth fabric weight.

8. The garment of claim 7, wherein the plurality of integrated compression zones further comprise:
a back top compression zone on the back side having the third amount of compression and the third fabric weight;
wherein the back top compression zone is between the right back side of the fourth compression zone and the left back side of the fourth compression zone and above the fifth back compression zone;
wherein the back top compression zone is directly or indirectly joined with the right back side of the fourth compression zone and the left back side of the fourth compression zone by a seamless transition, and wherein the back top compression zone is directly or indirectly joined with the fifth back compression zone;
wherein a difference between the third fabric weight and the first fabric weight ranges from 100 GSM to 220 GSM, wherein the first fabric weight is less than the third fabric weight.

9. The garment of claim 1, wherein the plurality of integrated compression zones further comprise:
a sixth compression zone having the second amount of compression and the second fabric weight,
wherein the sixth compression zone includes a sixth right compression zone and a sixth left compression zone, the sixth right compression zone is sized and shaped to cover at least a portion of an area corresponding to a wearer's front shoulder above the right breast area, the sixth right compression zone is above the first right compression zone, and the sixth left compression zone is sized and shaped to cover at least a portion of an area corresponding to a wearer's front shoulder above the left breast area, the sixth left compression zone is above the first left compression zone;
wherein the sixth right compression zone is directly or indirectly joined with the first right compression zone by a seamless transition, the sixth left compression zone is directly or indirectly joined with the first left compression zone by a seamless transition; and
wherein a difference between the second fabric weight and the first fabric weight ranges from 100 GSM to 220 GSM, wherein the first fabric weight is less than the second fabric weight.

10. The garment of claim 9, wherein each of the second right compression zone, the second left compression zone, the third compression zone, the fourth right compression zone, the fourth left compression zone, the fifth compression zone, the sixth right compression zone, and the sixth left compression zone each include the jacquard knit stitch construction.

11. The garment of claim 9, further comprising a right side strap and a left side strap, each of the right side strap and the left side strap extend from the front side to the back side, the right side strap comprises the sixth right compression zone, and the left side strap comprises the sixth left compression zone.

12. The garment of claim 7, wherein the fifth compression zone is sized and shaped to cover at least a portion of the mid-torso area of a wearer;
wherein the fourth compression zone is sized and shaped to cover at least a portion the mid-torso area of a wearer.

13. The garment of claim 7, wherein the fifth compression zone is sized and shaped to cover at least a portion of the mid-torso area and lower torso area of a wearer;
wherein the fourth compression zone is sized and shaped to cover at least a portion the mid-torso area and lower torso area of a wearer.

14. The garment of claim 9, wherein the plurality of integrated compression zones further comprise a fifth back compression zone, wherein the fifth back compression zone has the fifth amount of compression and the fifth fabric weight;
wherein the fifth back compression zone is sized and shaped to cover at least a portion of a back of the upper torso area, mid-torso area, and lower torso area of a wearer.

15. The garment of claim 1, further comprising a lower torso portion that is sized and shaped to cover at least a hip area and front pelvic area of a wearer.

16. The garment of claim 15, wherein the torso portion further comprises:
a right thigh compression zone that is sized and shaped to cover a front of the right thigh of the wearer, wherein the right thigh compression zone is directly or indirectly joined with the right side of the lower torso portion by a seamless transition; and
a left thigh compression zone that sized and shaped to cover a front of the left thigh of the wearer, wherein the left thigh compression zone is directly or indirectly joined with the left side of the lower torso portion by a seamless transition,
wherein each of the right thigh compression zone and the left thigh compression zone has a seventh amount of compression and a seventh fabric weight.

17. The garment of claim 1, wherein the torso garment is a shaper garment.

18. The garment of claim 1, further comprising:
a lining sized and shaped to cover at least a portion of the first right compression zone and the first left compression zone, wherein the lining is directly bonded to at least a portion of the first right compression zone and a portion of the left compression zone of the single engineered warp knitted component,
optionally, wherein the lining includes a right unbonded area and left unbonded area, wherein the lining in the right unbonded area includes a slit sized and shaped to allow insertion of a first pad into the right unbonded area between the first lining and the first right compression zone, wherein the lining in the left unbonded area includes a slit sized and shaped to allow insertion of a second pad into the left unbonded area between the lining and the first left compression zone.

19. The garment of claim 1, further comprising:
a first lining sized and shaped to cover at least a portion of the first right compression zone, wherein the first lining is directly bonded to at least a portion of the first right compression zone of the single engineered warp knitted component, and
a second lining sized and shaped to cover at least a portion of the first left compression zone, wherein the second lining is directly bonded to at least a portion of the first left compression zone of the single engineered warp knitted component;
optionally wherein the first lining includes a right unbonded area, wherein the first lining in the right unbonded area includes a slit sized and shaped to allow insertion of a first pad into the right unbonded area between the first lining and the first right compression zone, and wherein the second includes a left unbonded area, wherein the left lining in the left unbonded area includes a slit sized and shaped to allow insertion of a second pad into the left unbonded area between the lining and the first left compression zone.

20. The garment of claim 18, wherein a lower edge of the liner is curved forming a curved portion, the curved portion curving gently upwards towards a wearer's sternum area, and wherein a neck opening edge and an arm opening edge of the single engineered warp knitted component are folded inwards and bonded to a neck opening edge and an arm opening edge of the liner.
